SEQUENCE LISTING (7 sequences)

SEQ ID NO: 1 - protein, 69 aa, Artificial Sequence
  Vicrostatin
GDAPANPCCDAATCKLTTGSQCADGLCCDQCKFMKEGTVCRRARGDDLDDYCNGISAGCPRNPHKGPAT

SEQ ID NO: 2 - protein, 6 aa, Artificial Sequence
  Contortrostatin domain N-terminal
HKGPAT

SEQ ID NO: 3 - protein, 65 aa, Artificial Sequence
  CN disintegrin domain
DAPANPCCDAATCKLTTGSQCADGLCCDQCKFMKEGTVCRRARGDDLDDYCNGISAGCPRNPFHA

SEQ ID NO: 4 - DNA, 66 bp, Artificial Sequence
  Forward primer for rCN (disintegrin domain) and VCN (disintegrin domain)
gttccagatctcgagaatctttacttccaaggagacgctcctgcaaatccgtgctgcgatgctgca

SEQ ID NO: 5 - DNA, 51 bp, Artificial Sequence
  Reverse primer for rCN (disintegrin domain)
gttattcgccatggcttaggcatggaagggatttctgggacagccagcaga

SEQ ID NO: 6 - DNA, 66 bp, Artificial Sequence
  Reverse primer for VCN (disintegrin domain)
gttattcgccatggcttaagtagctggacccttgtggggatttctgggacagccagcagatatgcc

SEQ ID NO: 7 - protein, 7 aa, Artificial Sequence
  Canonical amino acid sequence
ENLYFQG

(19) United States
(12) Patent Application Publication
(10) Pub. No.: US 2016/0317610 A1
(43) Pub. Date: Nov. 3, 2016

(54) COMPOSITIONS AND METHODS FOR TREATING OVARIAN CANCER INCLUDING PREVENTING THE RECURRENCE THEREOF

(71) Applicants: Francis S. MARKLAND, JR., Manhattan Beach, CA (US); Stephen D. Swenson, Glendale, CA (US); Radu O. Minea, Arcadia, CA (US)

(72) Inventors: Francis S. MARKLAND, JR., Manhattan Beach, CA (US); Stephen D. Swenson, Glendale, CA (US); Radu O. Minea, Arcadia, CA (US)

(73) Assignee: University of Southern California, Los Angeles, CA (US)

(21) Appl. No.: 15/080,401

(22) PCT Filed: Sep. 25, 2014
(86) PCT No.: PCT/US14/57557

§ 371 Date: Mar. 24, 2016

Related U.S. Application Data
(60) Provisional application No. 61/882,517, filed on Sep. 25, 2013.

(51) Int. Cl. A61K 38/16; A61K 45/06

(52) U.S. Cl. CPC A61K 38/16; A61K 45/06

(57) ABSTRACT

A method of treating ovarian cancer (OC) includes administering to a subject in need thereof an effective amount of a pharmaceutical composition comprising vicrostatin and/or a protein substantially similar to vicrostatin. The administration is preferably performed intraperitoneally. The methods may include concurrently or sequentially administering to the patient one or more additional treatments for ovarian cancer. A pharmaceutical composition used in connection with the methods of the present invention comprise vicrostatin loaded in a viscoelastic gel comprising polyethylene oxide (PEO) and carboxymethyl cellulose (CMC).

### CROSS REFERENCE TO RELATED APPLICATIONS

This application is a national stage of international application no. PCT/US 14/57557, filed on Sep. 25, 2014 and claims the benefit of U.S. Provisional Application No. 61/882,517, filed Sep. 25, 2013, the entire contents of which are incorporated herein by reference in its entirety.

### FIELD OF THE INVENTION

The present invention relates to compositions and methods for treating and preventing ovarian cancer.

### BACKGROUND OF THE INVENTION

Ovarian cancer (OC) is a devastating disease with more than 14,000 deaths expected to occur in 2014 in the United States alone. The serous histotype is found in ˜70% of cases and its most aggressive, highly-invasive subtype, the high-grade serous ovarian cancer (HGSOC), accounts for 90% of serous carcinomas and two-thirds of all OC deaths. The standard treatment is aggressive surgery followed by adjuvant platinum-taxane chemotherapy. Most patients do respond initially to chemotherapy. Nonetheless, chemoresistant cancer recurs in about 25% of patients within 6 months and the overall 5-year survival is only ˜30%. A massive integrated genomic analysis of germline and somatic variants was recently conducted in HGSOC which generated high-resolution measurements of mRNA and microRNA expression, DNA copy number, and DNA methylation patterns from hundreds of clinical samples in conjunction with whole-exome DNA sequencing information also retrieved from most of these samples [1-4]. Overall, HGSOC shows a remarkable degree of genomic rearrangements which may be explained by the high prevalence of mutations/DNA promoter methylations in putative DNA repair genes, including homologous recombination components. While a high frequency of homologous recombination defects in HGSOC tumors may benefit from PARP inhibitors, the general absence of mutated/amplified oncogene targets (especially those for which therapeutic solutions already exist) is disconcerting as it significantly restricts the therapeutic options in HGSOC [5, 6]. On the other hand, the persistent activation of multiple components of the cellular invasion apparatus (i.e., invadosome-associated integrins and their downstream kinases) and the presence of cancer stem cell-associated markers, which globally correlate with invasiveness and chemoresistance, are consistent findings in HGSOC progression [7-10]. For these reasons, a more efficient and less toxic form of therapy aimed at addressing metastatic residual disease following surgery is urgently needed in order to prolong the survival and improve the quality of life of these patients.

The prevailing mode of OC dissemination is unique among solid tumors. Exfoliated OC cells from primary tumors as well as from macro and micrometastases assemble into compact, free-floating, highly invasive multicellular aggregates (referred to herein as “spheroids” or “OC Spheroids”) in peritoneal fluid that are carried away by the peritoneal fluid to secondary sites in the abdominal cavity where they attach, invade into submesothelial connective tissue and establish additional peritoneal micrometastases [11, 37 38]. Unlike primary tumors and macrometastases, OC micrometastases evade detection at the time of surgery, and failure to eradicate them is the main reason for recurrence [12]. These spheroids represent the main driving force behind secondary dissemination and OC recurrence.

The formation of these multicellular tumor aggregates that are held together by a complex but still poorly-understood fibronectin-rich tumorigenic extracellular matrix (ECM), is characteristically associated with poor prognosis and represents a highly adaptive mechanism that endows these cellular aggregates with protection from anoikis, allows for enhanced chemo-refractoriness and leads to the generation of highly dynamic actin cytoskeletons that are required for rapid spheroid dissemination, mesothelial layer clearance and submesothelial invasion [39, 40]. Several in vitro studies showed that OC spheroids use integrin-dependent activation of myosin motors to generate traction forces that displace mesothelial cells from underneath attached spheroids [40]. A key element to the initial assembly of OC spheroids consists of a complex cross-talk between oncofetal fibronectin isoforms and OC cells via multiple classes of integrin receptors [41]. Current evidence suggests that the interaction between vitronectin and fibronectin isoforms with αv and α5 integrins (αvβ3, α5β1, etc.) is required for the initial assembly of OC spheroids as well as for their prolonged survival as free-floating cellular aggregates in the ascites fluid and subsequent invasion [31, 42-45].

Several RGD-dependent integrins have been identified to be critically important to spheroid biology, suggesting that a broad pharmacological inhibition of tumoral integrins may represent a valid therapeutic strategy for prevention of secondary dissemination in OC. Integrins are heterodimeric cell surface adhesion receptors (containing α and β chains) operating at the interface between the ECM and cytoskeletal apparatus [49-51]. Several integrins recognize the RGD sequence present in several key ECM proteins [52] that are involved in development, angiogenesis and tumor progression. Integrins exhibit structural diversity and undergo conformational changes that are central to the regulation of receptor function. Integrins exist in three major conformational states: an inactive or low affinity state, a primed or active high affinity state, and a ligand bound or occupied state [49]. Active integrins are not usually displayed by quiescent tissues. Furthermore, integrins that are not normally expressed by most quiescent tissues, such as the αv and α5 members, play important roles in neoplastic processes including OC spheroid survival, serosal attachment, invasion (via invadosome formation) and angiogenesis [46, 53-56].

The cell-ECM interactions regulate the ability of cells to mechanically sense their environment by integrating multiple signaling pathways initiated by extracellular cues with the cell's cytoskeleton. Although integrins (including αvβ3, αvβ5, α5β1 and αvβ6) have important roles in OC cell attachment, survival, migration, invasion and angiogenesis (53-56), the precise roles played by different integrins in various aspects of tumor progression and why some integrins appear to be especially supportive of tumor progression are still not fully understood. The β3 integrin appears to be critically involved in regulation of pathological angiogenesis (83) although it doesn't seem to be essential for the formation of vasculature during development or physiological angiogenesis. The pharmacological blockade of integrin αvβ3 has been demonstrated to significantly reduce tumor angiogenesis in numerous cancer models and several drug candidates are currently in clinical trials (84). This integrin can also serve as an effective target for therapy directed at processes critical to tumor progression (adhesion, invasion and migration). Similarly, αvβ5 and α5β1 as well as a number of other integrins (notably α2β1, α4β1, and α6β4) have also been shown to play important roles in tumor angiogenesis, their pharmacological targeting by soluble ligands or monoclonal antibodies leading to reduced tumor microvessel density in various cancer models (85). These same integrins could also serve as effective targets for OC therapy. Integrin over-expression, mislocalization and dysregulated activity drives tumor progression; they are, therefore, attractive targets for cancer therapy (86-88), and particularly for a disintegrin-based therapy for OC.

Targeting integrins on ovarian cancer spheroids as a therapeutic strategy for reducing metastatic burden in was successfully demonstrated preclinically in the past [46]. IP treatment with volociximab, an α5β1-specific mAb, was shown to significantly reduce tumor burden, ascites formation and the number of metastatic foci, and to increase animal survival in OC xenograft models [45, 47]. Several integrin-targeted therapeutics have been evaluated in the clinic for advanced recurrent OC. However, both Cilengitide, an RGD peptide which specifically binds to αvβ3 and αvβ5 integrins, and volociximab failed to show clinical efficacy in advanced OC. Thus, there is a need for a therapy with clinical efficacy in advanced OC.

Disintegrins are a class of disulfide-rich peptides originally isolated from snake venoms, many of which contain a Arg-Gly-Asp (RGD) motif displayed at the tip of a flexible loop called the disintegrin loop [57-59]. These peptides hold a significant translational potential based on their high-affinity/high-specificity interaction with tumoral integrins and some desirable pharmacological attributes. The integrin-binding activity of disintegrins depends on the appropriate pairing of several cysteine residues responsible for the disintegrin fold, a mobile 11-amino acid loop protruding from the polypeptide core displaying the tripeptide motif RGD [60, 61]. Disintegrins bind ONLY to the active conformation of integrins on motile cells such as cancer cells and angiogenic endothelial cells [49, 58]. In vivo studies carried out by Markland and others have shown that disintegrins are well tolerated and can be infused without toxicity or detrimental effect on blood pressure, body temperature, or other physiological parameters [62, 63]. For instance, acute toxicity of disintegrins isolated to purity from snake venoms was studied in canine species by Markland et al. Disintegrin-treated animals did not exhibit changes in heart rate, EKG, or blood coagulation parameters, and showed no evidence of toxicity at the doses tested [64]. Subsequently, the Markland laboratory discovered a sequence-engineered RGD-disintegrin (i.e., VCN) that could be reliably produced in large quantity in a robust recombinant bacterial system [33]. In numerous studies carried out in mice, this synthetic disintegrin has been chronically administered with no visible side effects or signs of internal bleeding, indicating that mice tolerate the drug quite well [33].

Although still lacking standard-of-care status, IP therapy is currently regarded as an effective therapeutic modality for advanced OC by most clinical experts. In advanced OC a 21.6% decrease in the risk of death was reported in patients undergoing combined IP and IV therapy versus those undergoing IV therapy alone [13]. In 2006 the NCI issued a statement: “On the basis of the results of these randomized phase III clinical trials, a combination of IV and IP administration of chemotherapy conveys a significant survival benefit among women with optimally debulked ovarian cancer, compared to IV administration alone” [14]. However, when translating the NCI recommendation into clinical practice concerns arose about dosing, toxicity, and the proper techniques for surgical placement and access to the IP port [14, 15]. Due to the altered peritoneal fluid dynamics in OC post-debulking, drug clearance from peritoneal cavity is much slower than in normal subjects which results in a prolonged exposure of residual tumors and mesothelia to much higher drug concentrations compared to those achieved by IV administration [16-20]. Therefore, although efficacious in the long run, the regimens of currently used IP platinum and taxane agents are accompanied by dramatic off-target effects owing to the narrow therapeutic indices of the drugs themselves [21-23]. This is a main reason why a significant number of women opt out of the IP treatment despite its potential benefits [24]. Catheter related issues such as leakage, infection, or flow obstructions add to the problem and occur in up to 1 in 5 patients. Patients also experience abdominal distention, pain, and vaginal leakage due to the IP administration of large fluid volumes [25].

In summary, the current patient non-compliance associated with IP chemotherapy is the result of: toxic accumulation of chemotherapy agents in the IP space (metabolic imbalances and neurotoxicity); administration of large volumes of fluids along with chemotherapy (abdominal pain, discomfort, and nausea); long-term presence of indwelling catheters (infections and adhesions) [36]. Therefore, there is a strong need for alternative therapies.

### SUMMARY OF THE INVENTION

One object of the present invention is to provide an integrin-targeted therapeutic for the treatment of ovarian cancer, and especially for the treatment of advanced, recurrent ovarian cancer.

Another object of the present invention is to provide an integrin-targeted therapeutic that can concurrently and efficiently target multiple integrin pathways, for instance, both αv and α5 integrin pathways on OC spheroids and which can be administered intraperitoneally.

According to one embodiment, a method of treating ovarian cancer or preventing the recurrence of ovarian cancer comprises administering to a subject in need thereof an effective amount of a pharmaceutical composition comprising vicrostatin (VCN) or a polypeptide substantially similar to vicrostatin. Preferably, the pharmaceutical composition comprises VCN. When used in the treatment of ovarian cancer, vicrostatin and polypeptides substantially similar to vicrostatin selectively bind to tumors, resulting in fewer off-target effects than traditional ovarian cancer therapies and are characterized by potent broad-spectrum anti-invasive properties. Further, due to the high aqueous solubility of VCN, much smaller volumes of the drug are required for intraperitoneal administration, and the lower volumes should lead to improved patient compliance.

The pharmaceutical composition may further comprise saline or phosphate buffered saline or may comprise the protein loaded in a viscoelastic gel. In one embodiment, the viscoelastic gel comprises polyethylene oxide (PEO) and carboxymethyl cellulose (CMC).

The administration is preferably performed intraperitoneally, and may comprise concurrently or sequentially administering to the patient one or more additional treatments for ovarian cancer, wherein the one or more additional treatments does not include a disintegrin. Suitable addition treatments include performing a debulking surgery comprising a surgical incision permitting access to the peritoneal space of the subject, and administration of the pharmaceutical composition is performed after the debulking but before the close of the surgical incision. The additional treatments may also comprises one or more selected from the group consisting of chemotherapy, immune and radiation therapy.

In one embodiment, vicrostatin and polypeptides substantially similar to vicrostatin are loaded in a viscoelastic hydrogel preferably composed of polyethylene oxide and carboxymethyl cellulose for use in intraperitoneal (IP) administration for the treatment of ovarian cancer. These formulations provide the benefits of high-concentration local peritoneal treatment as well as sustained drug delivery. Additionally, these formulations prevent the formation of post-surgical adhesions, a significant source of morbidity during IP treatment. These formulations are used because of their high level of direct peritoneal exposure combined with beneficial low but effective systemic drug levels. These combined characteristics represent significant advantages over currently utilized systemic cisplatin- and carboplatin-based approaches. Drug administration via the viscoelastic gels does not depend on the large fluid volumes and catheters which are the reasons for the current intolerance of patients to IP-paclitaxel chemotherapy. Thus, the present invention overcomes a barrier for patients to comply with and continue on IP therapy. Importantly, abdominal pain, which limits current IP paclitaxel treatment, can be avoided by VCN delivery via Oxiplex, this approach not only guarantees slow drug release but also prevention of adhesions.

In one embodiment, a pharmaceutical composition comprises a disintegrin loaded in a viscoelastic gel comprising polyethylene oxide (PEO) and carboxymethyl cellulose (CMC), wherein the disintegrin comprises vicrostatin or a protein substantially similar to vicrostatin. Advantages of the viscoelastic gel delivery system include the slow release of VCN and related disintegrins. In addition, there is a selective binding to tumors, which results in lack of off-target effects. Another benefit is the prevention of adhesions, a significant problem following gynecological surgery. Therefore, therapy related complications can be minimized.

Other aspects and advantages of the invention will be apparent from the following description and the appended claims.

### BRIEF DESCRIPTION OF THE DRAWINGS

The file of this patent contains at least one drawing executed in color. Copies of this patent with color drawings will be provided by the Office upon request and payment of the necessary fee.

FIG. 1: SKOV3^{GFP/LUC} Tumor Bearing Mice Treated with Oxiplex/VCN or Oxiplex Alone. In this study SKOV3^{GFP/LUC} cells were injected IP and allowed to grow for two weeks. At this time treatment was initiated with VCN-Oxiplex (5 mg/ml, 1 ml administered once weekly, left 4 panels) or a control of Oxiplex alone (1 ml administered once weekly, right 4 panels). Tumor growth was followed by bioluminescent imaging weekly and representative animals after 2 and 4 weeks of treatment are shown. Animals were sacrificed after 4 weeks of treatment with extensive tumor growth observed in the control animals (black arrows) and no visible macroscopic tumor foci observed in treated mice.

FIG. 2: FIG. 2A-Comparison of Efficacy of VCN-Oxiplex to VCN-Saline in a SKOV3^{GFP/LUC} Spheroid Model of OC. SKOV3^{GFP/LUC} spheroids were implanted IP by direct injection in nude mice. Beginning on day 4, animals were treated with 5 mg VCN delivered in 1 ml of either Oxiplex or saline. Animals were imaged weekly via Xenogen imaging. The untreated control animals grow large tumors (red arrows) rapidly which spread throughout the peritoneum. The VCN-Oxiplex and VCN-Saline formulations greatly limited tumor growth and spread. FIG. 2B-Bioluminescent Quantitation of Imaged Tumors. The mice in the SKOV3^{GFP/LUC} spheroid model were imaged weekly beginning on day 4 post tumor implantation. To image the mice, the animals were anesthetized with isoflourane and the luciferin substrate (1 μg/g) injected. 60 sec following luciferin injection, the mice were placed in the Xenogen Ivis 200 optical imaging unit and images were acquired for 1 and 60 sec. For quantitation, the photon flux of bioluminescent areas was averaged and plotted over the time for each groups.

FIG. 3: Bioluminescent Images of Representative Mice after 4 Weeks of Treatment with either VCN-Saline or VCN-Oxiplex vs. Control. Representative 5 mice from larger groups are shown after 4 weeks of treatment. Beginning 4 days post spheroid implantation the mice were treated via IP injection with VCN either in saline or Oxiplex.

FIG. 4 shows the dissection and luciferase imaging of an ovarian tumor. Athymic female mice had 2.5×10⁵ OVCAR-31uc cells implanted into the intraperitoneal space via direct injection. The cells (OVCAR-31uc) stably express luciferase. Panel A shows a representative image of a dissected mouse with the yellow arrow indicating the location of tumor. Panel B is a Xenogen image of the luciferase secreting tumor, as the luciferase is secreted from the tumor cells the area of the tumor appears diffuse but correlates the dissected animal.

FIG. 5 shows the release of VCN from Oxiplex as determined by quantitative ELISA. VCN when impregnated in Oxiplex is release with an initial rapid release of ˜16% of the loaded material over the initial 48 hours. The gel remains intact for >11 days releasing nearly 55% of the loaded VCN.

FIG. 6 shows a side-by-side comparison of dissected mice bearing OVCAR-3 tumor. 2×106 OVCAR-3 cells were injected i.p. into nude mice. The control mouse were treated with Oxiplex only. In the test mouse, 10 mg/ml of VCN in 1 ml of Oxiplex was injected once per week. As shown in the figure, the control mouse exhibited widespread OC whereas the treated mouse showed no signs of OC spreading or bleeding.

FIG. 7 shows tumor growth in control mouse over 4 weeks period. The control mouse was injected with Oxiplex only. As shown in the figure, the tumor had grown considerably from 2 weeks to 4 weeks.

FIG. 8 shows the result of SKOV-3Luc mouse having been treated with 5 mg/ml VCN in Oxiplex over 4 weeks period. As shown in the figure, at the end of the same 4 week period, no visible tumor was observed in the treated mouse.

FIG. 9 shows a device for preparing VCN impregnated in a PEO-CMC gel.

FIG. 10. A flow cytometry analysis of integrin expression levels of various ovarian cancer lines. Five ovarian cancer cell lines (A2780, ES2, HEY, NIH-OVCAR3, and SKOV3) were stained with three different integrin antibodies (i.e., an alpha5 integrin specific antibody, a global alphav integrin antibody, and an alphavbeta3 specific integrin antibody) in order to assess the relative levels of integrin expression on these cell lines. This systematic analysis was conducted in an effort to match the integrin targets expressed by these various ovarian cancer lines with the specific set the broad-spectrum integrin antagonist VCN is known to bind to. Freshly harvested aliquots of each ovarian cancer lines were resuspended in PBS containing 10% FBS (106 cells/condition), mixed with either an alpha5 integrin specific antibody (clone IIA1, BD Pharmigen, San Diego, Calif.) or a global alphav integrin antibody (clone 272-17E6, Millipore, Billerica, Mass.) or an alphavbeta3 specific antibody (clone LM609, Millipore, Billerica, Mass.), incubated at 4° C. (or 37° C. in the case of the alphavbeta3 specific antibody) for 1 hr. An irrelevant isotype IgG antibody control was also included for each cell line. At the end of the incubation period, the cells were pelleted in a benchtop centrifuge, resuspended in fresh PBS containing 10% FBS, mixed with a PE-Cy5 labeled secondary (SCBT, Dallas, Tex.) and incubated at 4° C. for an additional 1 hr. Then the cells were again pelleted in a benchtop centrifuge, resuspended in fresh PBS plus 10% FBS, and further analyzed in a CyAn ADP FACS analyzer (Beckman Coulter, Brea, Calif.) equipped with a 488 nm solid-state laser source. All ovarian cancer lines stained positive with both the alpha5 specific and the global alphav antibodies indicating the widespread presence of these targets on all these lines. Except for the ES2 line, all the other ovarian cancer lines analyzed here stained negative with the LM609 alphavbeta3 specific antibody which suggests the most of these cell lines do not display a constitutively active alphavbeta3 integrin. Importantly, all antibodies used in this analysis are function blocking integrin antibodies that, similar to VCN, can only bind to the active conformations of these receptors. Similar to the data generated with Cy5-VCN (see the previous figure), the ES2 line has a distinctive staining pattern (i.e., two distinct subpopulations that stain differentially) with both the global alphav integrin antibody as well as with the alphavbeta3 specific one. The composite data in the Figure are representative histograms compiled from multiple independent (at least 3) flow cytometry experiments.

FIG. 11. Binding analysis of Cy5-labeled VCN (Cy5-VCN) to a panel of ovarian cancer cell lines. VCN was labeled with a Cyanine5 NHS ester probe (Lumiprobe, Hallandale Beach, Fla.) according to the manufacturer protocol. Freshly harvested aliquots of five ovarian cancer lines (A2780, ES2, HEY, NIH-OVCAR3, and SKOV3) were resuspended in PBS containing 10% FBS (106 cells/condition), mixed with Cy5-VCN (1/1000 dilution), and incubated at 4° C. for 1 hr in the presence of labeled disintegrin. At the end of the incubation period, the cells were pelleted in a benchtop centrifuge, resuspended in fresh PBS containing 10% FBS, and further analyzed in a CyAn ADP FACS analyzer (Beckman Coulter, Brea, Calif.) equipped with a red diode (643 nm) laser source. Unlabeled cells were used as negative controls for each cell line. The data in the figure are representative histograms from multiple independent (at least 3) flow cytometry experiments. All ovarian cell lines stained intensely with Cy5-VCN. According to the staining pattern, the ES2 cell line in particular, and to a lesser degree the SKOV3 cell line, appear to contain two distinct subpopulations that seem to stain differentially with the Cy5-labeled disintegrin.

### DETAILED DESCRIPTION OF THE INVENTION

A method of treating ovarian cancer according to one aspect of the present invention comprises administering to a subject in need thereof an effective amount of a pharmaceutical composition comprising vicrostatin (VCN) or a polypeptide substantially similar to vicrostatin. Preferably, the pharmaceutical composition comprises VCN.

VCN (vicrostatin) comprises the following amino acid sequence: GDAPANPCCDAATCKLTTGSQCADGLCCDQCKFMKEGTVCRRARGDDLDDYCNGISA GCPRNPHKGPAT (SEQ ID NO: 1) [101]. Methods of the present invention using VCN thus comprise administering a protein comprising SEQ ID NO: 1.

The amino acid sequence, structure and method of synthesizing VCN is disclosed, for instance, in U.S. Pat. No. 7,754,850, entitled “Chimeric Disintegrin Domain,” which issued Jul. 13, 2010, U.S. Pat. No. 8,110,542, entitled “Methods of expressing proteins with disulfide bridges,” which issued Feb. 7, 2012, and U.S. Pat. No. 8,338,365, entitled “Inhibiting integrin receptor binding with non-native monomeric disintegrin or monomeric disintegrin domains,” which issued Dec. 25, 2012, the entire contents of all of which are incorporated herein by reference in their entirety.

VCN is a recombinant chimeric protein that includes a contortrostatin domain N-terminal with a sequence HKGPAT (SEQ ID NO: 2) at the C-terminal end. “Contortrostatin” (CN) refers to a disintegrin isolated from Agkistrodon contortrix contortrix (southern copperhead) venom (Tiikha, Rote et al. 1994). CN is produced in the snake venom gland as a multidomain precursor of 2027 bp having a 1449 bp open reading frame encoding the proprotein, metalloproteinase and disintegrin domains. The precursor is proteolytically processed, possibly autocatalytically, to generate mature CN. The full length CN preprotein is encoded by the nucleotide sequence $5-1536 of the full length mRNA (GeneBank AF212305), whereas the disintegrin domain of CN represents 1339-1533 of the mRNA. The CN disintegrin domain, which contains 65 amino acids, is shown below.

(SEQ ID NO: 3)
DAPANPCCDAATCKLTTGSQCADGLCCDQCKFMKEGTVCRRARGDDLDDY
CNGISAGCPRNPFHA.

The tri-peptide motif RGD (Arg-Gly-Asp) in VCN is conserved in most monomeric disintegrins and is located at the tip of a flexible loop, the integrin-binding loop, which is stabilized by disulfide bonds and protruding from the main body of the peptide chain

Although the precise disulfide bond pattern in VCN has not been confirmed, all cysteine residues in VCN participate in disulfide bond formation (i.e., no free sulfhydryls are present in VCN). Also, two disulfide bridges are critical to the formation of the disintegrin loop (which displays the RGD tripetide motif) in the C-terminal half of the molecule. The of the disulfide bond pattern in contortrostatin (CN), the native disintegrin recombinant VCN was modeled after, can be inferred from that of similar native homodimeric disintegrins with known crystal structures and disulfide bond patterns, such as schistatin, which is a native homodimeric disintegrin purified from Echis carinatus. Echistatin, a small monomeric native disintegrin from which the HKGPAT sequence was used along with CN's in designing VCN, and is also purified from Echis carinatus (the saw-scaled viper).

The sequence alignment of contortrostatin and schistatin is as following:

*NSVHPCCDPVICEPREGEHCISGPCCENCYFLNSGTICKRARGDGNQD
YCTGITPDCPRNRYNV-schistatin
DAPANPCCDAATCKLTTGSQCADGLCCDQCKFMKEGTVCRRARGDDLDD
YCNGISAGCPRNPFH*-contortrostatin

Based on the information revealed from solving the crystal structure of Echistatin, the disulfide bond pattern of CN can be reasonably deduced as shown in the following image:

Contortrostatin (Homodimer, 65 Amino Acids Per Chain)

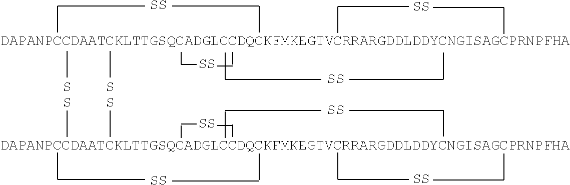

Please note that in CN two cysteine residues from each subunit participate in interchain disulfide bond formation (i.e., homodimerization), whereas the other eight in each chain are responsible for intrachain disulfide bond formation. Also as noted, two disulfide bridges are critical to the formation of the disintegrin loop (which displays the RGD tripetide motif) in the C-terminal half of the molecule. VCN, however, folds as a monomer and not as a homodimer like CN. Because all cysteine residues in VCN were shown to participate in disulfide bond formation (i.e., no free sulfhydryls are present in VCN), the reason VCN folds as a monomer is probably because the two cysteine residues that natively participate in interchain disulfide bond formation in CN do form novel (i.e., non-native) intrachain disulfide bonds in VCN. Despite this, the native configuration of the two disulfide bonds that are securing the disintegrin loop in CN and is responsible for the biological activity of the disintegrin, doesn't appear altered in VCN and is believed to be the same configuration as in CN.

A production method for a recombinant disintegrin, vicrostatin (VCN) has been published (91) and has also been described in U.S. Pat. No. 8,338,365. In brief, VCN can be reliably produced directly in the oxidative cytoplasm of Origami B E. coli (33). The pET32a vector can been used for expressing the desired peptide sequences fused downstream of a 109 amino acid thioredoxin wild type protein sequence (LaVallie, DiBlasio et al. 1993). Cloning sites are available for producing fusion proteins also containing cleavable His-tag sequence and S-tag sequence for detection and purification. As disclosed in the references cited herein, disintegrins including recombinant VCN sequences can be expressed as a fusion with thioredoxin to obtain an accelerated disulfide bond formation and an enhanced solubility of eukaryotic protein.

Contortrostatin wild type disintegrin domain or the disintegrin domain with SEQ ID NO: 2 C-terminal graft are directionally cloned by PCR into the pET32a vector, downstream of the thioredoxin sequence. The set of restriction enzymes used for cloning was: BglII/NcoI. The oligonucleotide primers employed for cloning were as follows:
CNfor1—forward primer for rCN (disintegrin domain) and VCN (disintegrin domain) introducing BglII restriction site

(SEQ ID NO: 4)
5′GTTCCAGATCTCGAGAATCTTTACTTCCAAGGAGACGCTCCTGCAAA
TCCGTGCTGCGATGCTGCA3′

CNback1—reverse primer for rCN (disintegrin domain) introducing the NcoI restriction site

(SEQ ID NO: 5)
5′GTTATTCGCCATGGCTTAGGCATGGAAGGGATTTCTGGGACAGCCAG
CAGA3′

CNback2—reverse primer for VCN (disintegrin domain) introduction the NcoI restriction site

(SEQ ID NO: 6)
5′GTTATTCGCCATGGCTTAAGTAGCTGGACCCTTGTGGGGATTTCTGG
GACAGCCA GCAGATATGCC3′

The forward primer introduces a TEV protease cleavage site, which makes possible the removal of the thioredoxin fusion partner after purification of the fusion protein by Ni-column chromatography. The TEV protease recognizes with high specificity the canonical ENLYFQG (SEQ ID NO: 7) amino acid sequence engineered between recombinant CN and the thioredoxin fusion partner in this construct and following cleavage leaves a glycine at the N-terminus of rCN and VCN. The reverse primer grafts the HKGPAT (SEQ ID NO: 2) segment to the C-terminus of the fusion protein. The recombinant fusion protein can be generated using the above described cloning strategy.

The vector is used to transform the expression host, Origami B(DE3)pLysS, for expression of rCN or VCN.

Without being limited to theory, VCN acts through a tumor targeted mechanism that involves high-specificity binding to invadosome-associated tumoral integrins (i.e., multiple αv and α5 members) displayed by OC [30-32]. This anti-invasive mechanism is experimentally supported by the inventors' previous studies with angiogenic endothelial and glioma cells [33, 34]. A soluble compact molecule, VCN disrupts and outcompetes (by 10-100-fold) critical interactions between oncogenic extracellular matrix (ECM) protein isoforms, such as fibronectins, vitronectin etc., and invading tumor cells thereby interfering with critical components of OC spheroid assembly and stability [34, 35].

Specifically, but without being limited to theory, VCN acts through multiple integrin ligation (i.e., αvβ3, αvβ5 and α5β1) on both endothelial and OC cells. There is a distinct advantage to the use of VCN and related disintegrins. VCN not only acts as an antagonist (in a manner identical to RGD peptides), VCN also elicits signaling responses acting as an agonist (33). Further, integrin αvβ3 is expressed at low levels on epithelial cells and mature endothelial cells, but is overexpressed on the endothelial cells of the tumor neovasculature and on tumor cells. Therefore, this integrin presents an attractive and tumor specific therapeutic target for rapidly growing solid tumors.

The complexity of the cancer microenvironment dictates that for optimal efficacy an anti-integrin therapeutic must target at least two members of the RGD-binding integrin class and preferably more [65]; VCN, which targets multiple αv and α5 integrin members, meets this criterion. Unlike cyclic RGD peptides and peptidomimetics, VCN has additional structural elements which enable it to modulate integrin signaling in an efficient and unique manner [66-71]. Thus, unlike Cilengitide or cyclo(L-Arg-Gly-L-Asp-D-Phe-N-methyl-L-Val), a cyclic RGD peptide developed by Merck KGaA, the RGD-loop in VCN has additional flanking residues, enabling it to make more extensive contacts with the receptor. Also, NMR and crystallographic studies have revealed that the C-terminal tail in disintegrins folds with the RGD-containing disintegrin loop, such that these two structural elements are linked together in an extended conformational epitope, suggesting these two functional regions are engaged in extensive interactions with the target integrin receptor [67, 68]. This finding is further supported by the inventors' work on the engineering of the COOH-terminus of VCN, which resulted in an improved affinity for α5β1 though a rational design [33]. By using structural and functional regions in addition to the RGD motif, a sequence that serves as the sole basis for the design of cyclic RGD peptides and RGD mimetics, disintegrins exhibit novel antitumor activities (such as actin cytoskeleton disruption via receptor triggering) compared to small cyclic RGD peptides and peptidomimetics.

Although VCN is the preferred disintegrin used in connection with the ovarian cancer treatment methods of the present invention include the use of a disintegrin polypeptide that is substantially similar to VCN. Methods of the present invention using polypeptides substantially similar to VCN comprise administering a protein comprising a sequence substantially the same as SEQ ID NO: 1. In this context, a sequence “substantially the same” refers to nucleic acid or amino acid sequences having sequence variation that do not materially affect the nature of the protein (i.e. the structure and substrate specificity and/or biological activity of the protein). With respect to amino acid sequences, the amino acid substitutions should be generally conservative substitutions and/or variations in regions of the polypeptide not involved in determination of structure or function. For instance, in one embodiment, a polypeptide can be considered “substantially similar” to vicrostatin so long as the selected disintegrin binds to the αv and α5 integrin members, retains the tri-peptide motif RGD (Arg-Gly-Asp) located at the tip of an integrin-binding loop, protrudes from the main body of the peptide chain and is stabilized by a suitable number of disulfide bonds. The number of stabilizing disulfide bonds in the disintegrin is preferably at least three, more preferably at least four, and preferably all of the disulfide bonds in VCN. One example of polypeptide substantially similar to VCN is contortrostatin.

Vicrostatin and polypeptides substantially similar to vicrostatin may be used individually or in combination for the treatment of ovarian cancer. Thus, a method of treating ovarian cancer according to another aspect of the present invention comprises administering to a subject in need thereof an effective amount of a pharmaceutical composition comprising one or more disintegrin, wherein the disintegrin comprises one or more selected from the group consisting VCN and a polypeptide substantially similar to VCN. Here, the VCN and the polypeptides substantially similar to VCN are used individually, or in combination, for the treatment of ovarian cancer.

VCN and the polypeptides substantially similar to VCN used in connection with the present invention are preferably administered in the form of pharmaceutical compositions. Compositions and formulations for parenteral administration, and particularly intraperitoneal administration, preferably comprise lyophilized formulations or aqueous solutions. Pharmaceutical compositions suitable use in connection with the present invention are generally prepared by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, ordextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration should generally be sterile. This is readily accomplished by filtration through sterile filtration membranes, prior to or following lyophilization and reconstitution.

In a preferred embodiment, the disintegrins of the present invention, including VCN, are preferably lyophilized and dispersed, dissolved or suspended in a saline solution or in phosphate buffered saline (PBS) solution. The concentration of the disintegrin in the solution to be administered is limited only by the solubility of the disintegrin in the saline or phosphate buffered saline. Concentrations of the disintegrin in the saline or phosphate buffered saline pharmaceutical composition may range from 0.001 mg/mL (mg disintegrin/total volume of composition) to about 50 mg/ml, or 0.5 mg/ml to 40 mg/ml, or 1 mg/ml to 20 mg/ml. Specific examples, include 1 mg/ml, 2 mg/ml, 5 mg/ml and 10 mg/ml.

VCN and the polypeptides substantially similar to VCN used in connection with the present invention are preferably administered in the form of a sustained release formulation. Thus, one aspect of the present invention is directed to a pharmaceutical composition that comprises VCN or a polypeptide substantially the same as VCN loaded in a viscoelastic and physiologically acceptable gel. Preferably, the viscoelastic gel is a gel comprising polyethylene oxide (PEO) and carboxymethyl cellulose (CMC) (a “PEO/CMC gel”) such as those described in U.S. Pat. No. 5,156,839, entitled “Covering wound with composition of carboxymethyl cellulose and polyoxyethylene oxide,” which issued Oct. 20, 1992 and/or U.S. Pat. No. 5,906,997, entitled “Bioresorbable compositions of carboxypolysaccharide polyether intermacromolecular complexes and methods for their use in reducing surgical adhesions,” which issued Mar. 25, 1999, the contents of both of which are incorporated herein by reference in their entirety. A method for treating ovarian cancer in accordance with this aspect thus comprises administering to a patient in need thereof an effective amount of a pharmaceutical composition comprising the selected disintegrin loaded in a PEO/CMC gel.

Suitable PEO/CMC gels useable in connection with the present invention include those sold by FzioMed Inc., such as “Oxiplex/AP” and sometimes referred to herein simply as “Oxiplex” (trade name Intercoat™, FzioMed San Luis Obisbo, Calif., distributed in at least some jurisdictions by Ethicon, Inc.). Oxiplex/AP is a viscoelastic gel composed of polyethylene oxide and carboxymethyl cellulose stabilized by calcium chloride [26, 27] and is currently approved for clinical use for prevention of post-surgical adhesions in Europe and Asia [28, 29]. It is presently undergoing FDA approval in the U.S. for a similar application. Oxiplex/AP Gel is specifically formulated for laparoscopic application, with tissue adherence and persistence sufficient to prevent adhesion formation. One advantage of using the Oxiplex/AP as a delivery vehicle is that VCN is released slowly over time while the delivery system has the advantage of preventing post-surgical adhesions in ovarian cancer debulking, a common post-operative occurrence.

PEO/CMC gels useable in connection with the present invention are generally prepared by combining a solution of VCN (or a disintegrin substantially similar to VCN) with the selected PEO/CMC gel. The methods for combining the disintegrin and the PEO/CMC gel is not particularly limited. The VCN may be loaded in a PEO/CMC gel by suspending the desired amount of VCN in a volume (<50 μl) of PBS (phosphate buffered saline), saline or otherwise pharmacologically acceptable solvent and mixing the VCN suspension with the PEO/CMC gel until the disintegrin is suitably dispersed in the PEO/CMC gel, preferably as a homogenous suspension of disintegrin in the PEO/CMC gel. Once suitably mixed, and where appropriate sterilized, the resulting pharmaceutical composition can be administered.

In preparing the disintegrin and PEO/CMC gel formulations, it is preferred that a very small volume of a highly concentrated disintegrin solution is mixed with the PEO/CMC gel, so that the gel is not diluted by more than 5%. It was found that by diluting PEO/CMC gel with larger volumes of disintegrin solution, regardless of the concentration of protein, the gels became structurally unstable and when diluted with a drug volume representing more than ˜20% volume of the gel, the gel fails to retain VCN. Preferably, when preparing the VCN impregnated PEO/CMC gel, and specifically Oxiplex/AP, a volume of the PEO/CMC gel is diluted less than 20% by a volume of the disintegrin solution, and preferably diluted less than 5% by a volume of the VCN solution.

The concentration of the disintegrin in the PEO/CMC gel is not particularly limited so long as the gel releases the disintegrin in a sustained release manner. This can be determined according to the methods described herein. Preferably, the disintegrin loaded in the PEO/CMC gel can be released from the gel over a time period of at least two days, more preferably at least three days, more preferably at least 4 days, and more preferably at least a week. In a preferred embodiment, VCN can be incorporated into Oxiplex/AP at concentrations of 2-10 mg/ml (total volume) and resulted in a sustained release of the drug that lasted for up to 10 days.

In order to optimize gel formulation in the intraperitoneal space, gel formulations that deliver the optimal dose and rate of release under flow conditions can be used. For instance, the gel can be placed in a mesh cage and placed in a circulating flow stream that has 10% of its volume replaced daily. All material can be collected for each day's removal and the samples subjected to the above described ELISA to determine normalized rate and amount of release. VCN released over a period of days to weeks from the Oxiplex gel with full potent bioactivity can be established in order to identify the formulation that will deliver therapeutic composition in a desired manner.

If PEO/CMC gels do not release the disintegrin as needed in a particular application, a number of alternative disintegrin delivery strategies could be utilized. For instance, the disintegrin may be encapsulated in copoly(lactic/glycolic acid) (PLGA) pellets. The pellets can formed through lyophilization of organic solvents from a mixture containing different percentages of PLGA and a constant amount of disintegrin. In the case of CN, it was observed that using 24% PLGA we were able to encapsulate CN and maintain first order release over 28 days. Due to their inherent stability CN and VCN retain biological activity after encapsulation. CN released from the pellets was evaluated for inhibition of platelet aggregation and found to have an IC₅₀ identical to that of native CN.

The sustained release formulation may be formulated weeks, months or even years in advance of the administration of the pharmaceutical composition. Alternatively, the sustained release formulations may be made in the same week, on the same day as or even immediately before administration to the subject. As such, the methods of the present invention may optionally include preparing the pharmaceutical composition by, for instance, combining a solution of VCN (or a disintegrin substantially similar to VCN) with the selected viscoelastic gel as set forth herein.

Subjects who may benefit from or are in need to the treatment methods of the present invention are those who have been diagnosed with ovarian cancer, including those who have been diagnosed with or are at risk for developing high-grade serous ovarian cancer (HGSOC).

As used herein, the phrases “treating ovarian cancer,” “treatment of cancer” or “preventing ovarian cancer” means to have one or more of the following effects: to inhibit the inhibit the formation or spread of primary tumors, macrometastases or micrometastases, decrease the size of macrometastases, inhibit the formation of the OC Spheroids, lessen or reduce the number of OC Spheroids, to antagonize the Epithelial-Mesenchymal transition invasion program of ovarian cancer cells, or ameliorate or alleviate one ore more symptoms of the disease caused by the ovarian cancer. An “effective amount” of the pharmaceutical compositions of the present invention is an amount sufficient to carry out a specifically stated purpose. An “effective amount” may be determined empirically and in a routine manners in relation to the stated purpose. Inhibiting or reducing the formation, size or spread macrometastases, micrometastases, or OC Spheroids may be shown either by the absence in relation to untreated controls.

Further, the ability of VCN and polypeptides substantially similar to VCN to target pathways critical to spheroid formation provide a mechanism for preventing the recurrence of ovarian cancer. Among the pathways targeted by VCN and the polypeptides substantially similar to VCN, multiple αv members as well as α5β1 have been shown to be critical to spheroid formation.

As OC progresses, tumor cells are believed to activate an EMT (epithelial-mesenchymal transition) process which allows them to either individually detach and then assemble into spheroids or collectively detach (i.e., spontaneous spheroid budding) from advanced primary tumors, then spread as free-floating spheroids carried by the dynamics of the peritoneal fluid, and invade into secondary abdominal sites. While most macrometastases that are spawned by these initial metastatic foci will form visible deposits and will be accessible for surgical removal during debulking surgery, a number of micrometastases will inherently be left behind after surgery. Some of the micrometastases will eventually stop responding to adjuvant chemotherapy and become responsible for disease recurrence and dissemination. A locally-delivered (IP) anti-invasive agent represents a critical component in addition to adjuvant chemotherapy for successful delay and possibly complete inhibition of OC dissemination.

A number of mesenchymal integrin subclasses (i.e., the αv and α5 integrins) are key components of the EMT processes executed by OC spheroids as they are transitioning to an invasive phenotype. VCN and polypeptides substantially similar to VCN, which binds to multiple αv and α5 integrin subclasses successfully antagonize (e.g., disrupt or inhibit) the EMT process executed by the spheroids. Intraperitoneal delivery of VCN, preferably in combination with standard-of-care agents administered systemically efficiently targets the OC cells as they are executing an EMT invasive process and prevent disease recurrence and further dissemination. Another aspect of the present invention is a method of preventing the recurrence of Ovarian cancer comprising administering to a patient in need thereof an effective amount of a pharmaceutical composition comprising vicrostatin and/or polypeptides substantially similar to vicrostatin. Here, vicrostatin and/or polypeptides substantially similar to vicrostatin are used to prevent the recurrence of ovarian cancer.

The use of disintegrins for OC therapy delivered IP via a carboxymethyl cellulose (CMC) polyethylene oxide (PEO) gel should optimize drug delivery and minimize any systemic side effects.

In the methods of treating ovarian cancer according to the present invention, the pharmaceutical compositions disclosed herein are preferably administered intraperitoneally. Intraperitoneal administration generally refers to delivery of the pharmaceutical composition into the peritoneum (the body cavity) by for instance, injection or via a catheter. The intraperitoneal administration of the stable soluble disintegrin peptides of the present invention interferes with multiple integrin pathways utilized by OC spheroids. The inventors' in vivo data generated with xenografts established via IP spheroid implantation indicate that VCN exhibits exceptional anti-invasive activities when the drug is delivered intraperitoneally.

The dosage administered should generally be sufficient to produce the desired effect of the treatment and preferably minimizes side effects and toxicity. Preferably, the dose is below the level where significant toxicity occurs. Preferably, the dosage administered is less than 3 mg of VC/kg of subject body weight, or less 10 mg/kg, or less than 25 mg/kg or less than 75 mg/kg. The dosage may be administered during a single intraperitoneal administration. Alternatively, the dosage may be divided amongst several intraperitoneal administrations during a treatment time period. For intraperitoneal administration, a dosage may be divided over two or more administrations during a treatment time period, such as a week.

The disintegrin of the present invention may be periodically re-administered throughout an entire time period during which the desired effect is desired to be maintained. For instance, the disintegrin may be periodically re-administered throughout the entire time period for which the subject has clinically observable primary tumors or macrometastases, or is at risk for the formation of primary tumors, macrometastases or micrometastases, is diagnosed as forming OC Spheroid or is at risk for OC Spheroid formation. For instance, the disintegrin may be periodically administered throughout the time period during which the subject is undergoing chemotherapy with other chemotherapeutic agents, radiation therapy, immune therapy or PARP therapy. The disintegrin may be re-administered periodically over a period of 1 month, or two months, or 6 months, a year, or two years, or even over the entire lifetime of the subject.

Although intraperitoneal administration is preferred, the pharmaceutical compositions of the present invention may also be administered parenterally, and especially intravenously.

The methods of treating ovarian cancer according to the present include administering to a patient in need thereof an effective amount of a pharmaceutical composition comprising a disintegrin, especially vicrostatin, in combination with at least one other procedure or treatment for ovarian cancer. Current methods of treating ovarian cancer include surgery including debulking surgery, chemotherapy, radiation therapy and immune therapy. The combination treatment may be performed sequentially or concurrently with the administering of pharmaceutical composition containing the disintegrin, especially VCN. This anti-invasive activity of VCN combined with surgery and/or systemic chemotherapy, radiation therapy or immune therapy provides for the treatment of ovarian cancer a mechanism for inhibition of tumor growth and dissemination.

The standard treatment for ovarian cancer is currently surgery (for diagnosis, staging and tumor debulking) followed by chemotherapy. In laparotomy, an incision is made in the abdomen, the area is examined, cancerous tissue is removed, and if necessary, fluid is drained from the abdominal region.

During laparotomy, tumor debulking is performed. In the debulking procedure, the ovaries as well as the uterus, cervix and fallopian tubes may be removed, in addition to as much visible disease as possible, with the goal of leaving no tumor nodule behind that measures more than a specified size (e.g. one centimeter). For instance, if a malignant tumor is found beyond the female reproductive system organs, portions of the diaphragm, bowel, spleen, and/or liver may be removed if the cancer has invaded and spread into these areas. However, for women who want to have children, if the cancer is at a very early stage it is sometimes possible to remove only the affected ovary (unilateral oophorectomy) and its adjoining fallopian tube (unilateral salpingectomy). The pharmaceutical compositions of the present invention may be directly applied in the intraperitoneal space to the internal organs and surfaces during the laparotomy after the debulking using, for instance, a cannula attached to a syringe. Further, when a pharmaceutical composition comprising the disintegrin loaded in a PEO/CMC gel is used, the gel may applied or sprayed directly onto the surfaces after tumor de-bulking and before the patient is closed.

A second or subsequent debulking operation may be beneficial for some women with recurrent ovarian cancer, depending on how long they were disease free and in how many sites the cancer recurred. Where a second or subsequent debulking operation is performed, the pharmaceutical compositions of the present invention may be directly applied in the intraperitoneal space to the internal organs and surfaces during the laparotomy after the de-bulking using, for instance, a cannula attached to a syringe. Further, when a pharmaceutical composition comprising the disintegrin loaded in a PEO/CMC gel is used, the gel may also applied or sprayed directly onto the surfaces after tumor debulking and before the patient is closed.

Common chemotherapy drugs currently used to treat ovarian cancer include cisplatin or carboplatin, and paclitaxel or docetaxel, which may be given in combination. For ovarian cancers that have recurred or returned, the patient may be prescribed topotecan, liposomal doxorubicin, etoposide, gemcitabine, vinorelbine, cyclophosphamide. The pharmaceutical compositions of the present invention may be administered concurrently or sequentially with these chemotherapy drugs.

Radiation therapy may be given over a period of several weeks. It is rarely used as a primary treatment for ovarian cancer, but is sometimes considered after the removal of a recurrent tumor or in the treatment of a recurrence. The pharmaceutical compositions of the present invention may be administered concurrently or sequentially during the course of radiation therapy.

The intraperitoneal administration of the disintegrin according to the present invention may be combined with the administration of one or more PARP (poly-ADP-ribose polymerase) inhibitors. Typically, PARP enzymes inside a cell repair damage to the cell's DNA. By stopping PARP activity in cancer cells, researchers may be able to prevent this repair so that cancer cells die off. In early studies, PARP inhibitors have been shown to work well in women with BRCA1 or BRCA2 mutations and ovarian cancer.

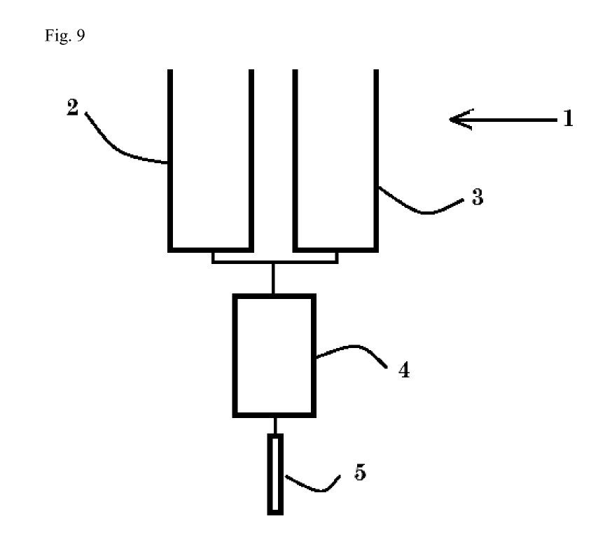

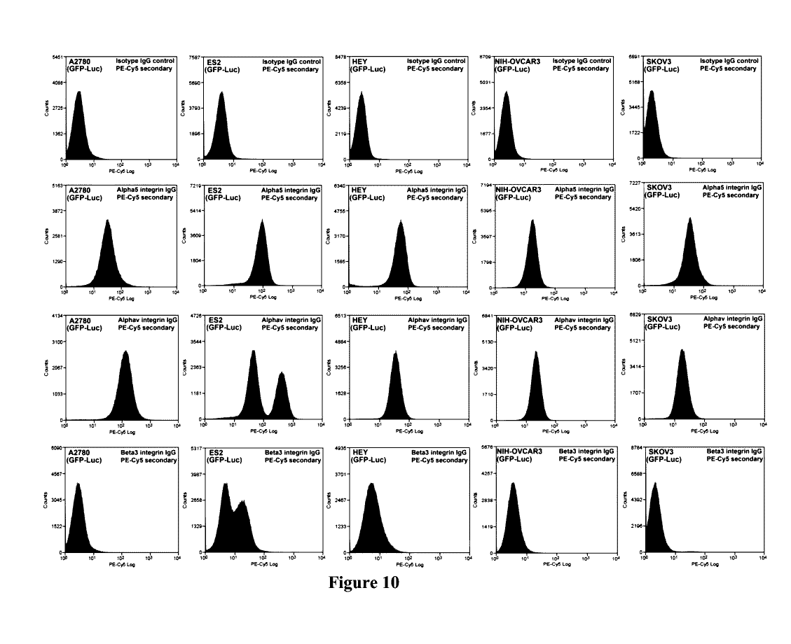

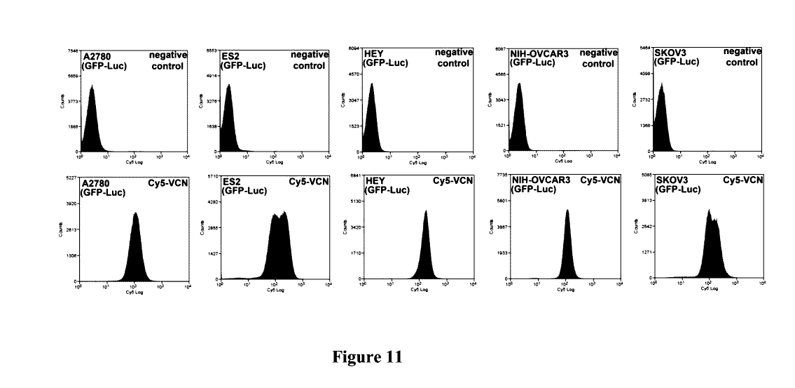

Another aspect of the present invention is directed to a kit for treating OC that includes a device for preparing vicrostatin (VCN) loaded in a polyethylene oxide (PEO) and carboxymethyl cellulose (CMC) gel. The kit for treating ovarian cancer comprises: a first enclosed container comprising a sterile solution of vicrostatin; and a second enclosed container comprising a sterile viscoelastic gel comprising polyethylene oxide and carboxymethyl cellulose; and written instructions for combining the contents of the two containers to form a vicrostatin loaded gel for the treatment of ovarian cancer. A device (1) for preparing a pharmaceutical composition of the present invention comprises as shown in FIG. 9, includes: a first chamber (2) having a solution of VCN, a second chamber (3) having a PEO/CMC gel, a mixing chamber (4) for mixing the VCN with the PEO/CMC gel, thereby forming a pharmaceutical composition for the treatment of ovarian cancer. The mixing chamber is operably coupled to the first and second chambers. A delivery member (5) is operably coupled to the mixing chamber for delivering the VCN-PEO/CMC gel. The disintegrin in the kit and/or device may be substituted for or may include a polypeptide substantially the same as VCN.

The following examples are provided in order to demonstrate and further illustrate certain embodiments and aspects of the present invention and are not to be construed as limiting the scope thereof.

### EXAMPLES

### Materials, Methods and Models

Animal Model.

Improvement of ovarian cancer patient treatment and outcome requires well-characterized animal models in which to evaluate novel therapeutics (93). Animal models using small laboratory animals are advantageous because they allow use of human ovarian cancer cell lines or tissues, which mimic the true disease process and can be manipulated by novel therapeutic compounds or drug administration strategies. The drug induced tissue response can be quantified and monitored by the clear-cut parameter of increased survival (94). Previously the inventors have established and validated an ovarian cancer mouse model based on injection of immortalized human OC cells into the peritoneal cavity of immunocompromised mice mimicking the pathogenesis of recurrence due to carcinomatosis (95). One of the cell lines that was investigated was the well characterized A2780^{SEAP} human ovarian adenocarcinoma cell line (obtained from Tom Hamilton/Fox Chase Cancer Center). Accurate and continuous determination of tumor burden in tumor bearing animals is often problematic. Necropsy of the mice would allow an accurate assessment of tumor volume at a specific time point. But this would require large experimental animal groups to study survival effectively. Therefore, tumor cells that express the surrogate marker heat-stable alkaline phosphatase (SEAP) due to stable vector transfection were utilized. SEAP is secreted by the tumor cells and can be quantified in the blood of animals after endogenous alkaline phosphatase is eliminated by heating the sample. Since SEAP plasma levels truly correlate with tumor cell load in animals, quantification of this surrogate marker represents a unique in vivo approach for continuous measurement of tumor cell burden in the study mice (95). As shown by Bao (95) transfection of the cells with the SEAP vector did not affect histopathology, morphology, or tumor progression and remained stable until animals succumbed to the disease after 40 days. The inventors used the A2780^{SEAP} model in studies to test the efficacy of liposomal disintegrin, contortrostatin (LCN), on growth and spread of human ovarian cancer cells. After day 12, LCN treated animals showed significantly lower levels of SEAP than controls. The improved treatment outcome with LCN correlated with gross observation of tumor upon necropsy (96).

Ovarian Cancer Spheroid Model of Tumor Growth.

The spheroid model of OC growth is increasingly regarded as a more physiologically relevant model. Therefore, once an optimally efficacious dose for VCN-Oxiplex formulation was identified, it was decided to test this dose in a spheroid model of SKOV3^{GFP/LUC}. Previous in vivo models employed the injection of a large number of dissociated cells harvested from OC cell monolayers grown in culture. Spheroids are multicellular aggregates that can be easily grown in vitro from any human OC cell line by simply seeding a defined number of individual cells in appropriate media and forcing the cells to grow in suspension by plating them on extremely low binding surfaces. To generate spheroids from SKOV3^{GFP/LUC} cells, multi-well plates were coated with poly(2-hydroxyethyl methacrylate) or polyHEMA onto which were seeded a defined number of cells in their regular medium and these were allowed to form spheroids over a defined time interval. Briefly, 1×10⁶ individual cells were seeded per well (in 6-well plates) and allowed to aggregate for at least 48 hours. Then the spheroids were resuspended at a density of 20×10⁶ cell equivalents/ml based on the initial number of individual cells that were initially seeded into each well. Spheroid inocula of 2×10⁶ cells in 0.1 ml saline were then injected into the peritoneal space of nude mice using a large bore needle. As a control for spheroid growth a group injected with inocula consisting of 2×10⁶ standard dispersed cells prepared as described in previous studies were included. Following inoculation, the animals were imaged weekly to follow tumor growth and spread. The bioluminescent optical imaging revealed that unlike dissociated cells which form a smaller number of macroscopic foci, the inoculated spheroids reproducibly form significantly higher numbers of smaller size tumor foci starting as early as 4 days post inoculation, which is consistent with a much different tumor growth behavior compared to when dissociated cells are used as an initial inoculum. The spheroids were allowed to grow until the animals progressed to sacrifice criteria, reached in about 30 days from the time of inoculation.

### Example 1

### Preparation of VCN-Impregnated Viscoelastic Gels

Formulations of VCN-impregnated Oxiplex gels for in vitro evaluation of VCN release were prepared and evaluated. In these experiments, identical volumes of Oxiplex (1 ml) impregnated with 1, 3 or 10 mg/ml VCN were used and it was determined that the release kinetics varied inversely proportional with drug concentration (i.e., 10 mg/ml being the slowest). For these experiments, a very small volume of VCN (<50 μl) of different drug concentrations was mixed with the Oxiplex, ensuring that the gel was not diluted by more than 5%. A representative procedure for preparing VCN impregnated in a PEO/CMC gel (here, Oxiplex) is as follows:
1. The desired amount of VCN is lyophilized and resuspended in a volume (<50 μl) of PBS (phosphate buffered saline) or saline, in this case the VCN was 1% final volume in 5 ml of gel.
2. The VCN is placed in a syringe (5 ml or greater to prepare 5 ml of gel) and attached through a pass through syringe coupler to a syringe of equal or greater size containing Oxiplex.
3. The contents of the syringes are mixed by moving the Oxiplex gel into the syringe containing VCN back and forth a minimum of 25 times to ensure a homogenous suspension.
4. The material is then moved to one of the two syringes and is ready for administration.

### Example 2

### Release Kinetics of VCN from Oxiplex

A number of formulations of VCN-impregnated Oxiplex gels were prepared and tested for in vitro evaluation of VCN release. In these studies, identical volumes of Oxiplex (1 ml) impregnated with an amount of 1, 3 or 10 mg of VCN were used and it was determined that the release kinetics varied inversely proportional with drug concentration (i.e., 10 mg/ml being the slowest). For these studies, a very small volume of VCN (<50u1) of different drug concentrations was mixed with the Oxiplex, ensuring that the gel was not diluted by more than 5%. In additional studies it was found that by diluting Oxiplex with larger volumes of VCNsubst, regardless of the concentration of protein, the gels became structurally unstable and when diluted with a drug volume representing more than ˜20% volume of the gel, the gel fails to retain VCN.

Further studies involved the use of different buffers or solutions as the diluent for VCN and from these studies it was found that PBS or saline was a superior diluent compared to organic solvents such as DMSO or isopropanol etc. All these experiments were repeated at least 3 times with reproducible results. From these studies, it was concluded that VCN incorporated into Oxiplex at 2-10 mg/ml (weight per volume) resulted in a sustained release of the drug that lasted for up to 10 days. An in vivo evaluation study with VCN-Oxiplex was conducted. In this study, NIH-OVCAR-3 cells (2×10⁶) were injected IP in nude mice and two weeks were allowed for tumors to develop before VCN-Oxiplex therapy was initiated. The therapy was administered for 4 weeks. The animals received once weekly either 1 ml Oxiplex alone (control group) or 1 ml Oxiplex impregnated with VCN at 10 mg/ml (treatment group). At the end of the study, the animals were dissected with the control group showing widespread tumor foci throughout the peritoneal cavity, while VCN-treated animals being devoid of macroscopic tumors on visual inspection. Importantly, there was no sign of internal bleeding in the treated animals.

### Example 3

### Dose Response/Frequency Studies VCN-Oxiplex

In the original animal model, the NIH-OVCAR3 cell line was used. However, in subsequent studies the inventors switched to the somewhat more aggressive but also more studied SKOV3 model for which a massive amount of data was already generated and reported in the literature. Before initiating these studies, the SKOV3^{GFP/LUC} cells stably infected with a lentivirus construct expressing a tandem of reporter genes: luciferase and GFP were prepared. Once infected, these cells were expanded and subjected to multiple rounds of FACS sorting to select for and enrich in cell populations expressing high levels of reporter genes. In parallel, a dose-response study with VCN-Oxiplex using wild-type SKOV3 cells was completed. This study helped with the understanding of the behavior of this tumor model. In these studies, the effects on tumor dissemination of 1, 2.5 and 5 mg VCN delivered weekly embedded in 1 ml Oxiplex versus Oxiplex alone were evaluated. All animals were sacrificed when the control animals reached the endpoint set for the study (i.e., excessive tumor growth with ascites buildup, visible signs of emaciation, and a loss of 10% or more of body weight). At the conclusion of the study, a dose dependent effect of VCN-Oxiplex administration which was determined subjectively by visual scoring of the number of macroscopic tumor foci (tumor foci visible by naked eye inspection also known as macrometastases) was observed. The control animals showed upon careful dissection extensive and widespread macroscopic carcinomatosis throughout the peritoneal cavity, while the treated groups showed a significant decrease in the total number of macroscopic foci that correlated with the administration of increasing amounts of drug (e.g., the animals that received 5 mg/ml of VCN-Oxiplex weekly showed the least amount of tumor spread). The next step was to determine how does the dose of 5 mg of VCN delivered in 1 ml of Oxiplex once weekly compare in terms of efficacy with the same amount of drug administered in multiple smaller doses weekly. Daily, every other day, and twice a week administrations of smaller fractions of the total amount of 5 mg/week were evaluated. Importantly, these and all our subsequent efficacy studies were conducted with SKOV3^{GFP/LUC} cells. In the daily and every other day regimens, the repeated administrations of the viscous Oxiplex gel via a large bore needle created wounds in the abdominal walls which resulted in local tumor invasion and colonization with multiple tumor foci becoming visible under the abdominal tegumentum of these animals. The conclusion of this study was that a dose of 5 mg of VCN administered once weekly is more efficacious than the same dose divided in multiple smaller doses and administered more frequently (Table 1).

TABLE 1
Evaluation of VCN-Oxiplex Dose Regimen

| | | | Tumor | |
|---|---|---|---|---|
| | | | Growth | |
| Formulation(a) | | | Imaging/ | Clinical |
| VCN mg/ml | Dose | Frequency | Visual | Relevance |
| Control | 1 ml | 1x/week | ++++/++++ | Control |
| Oxiplex Alone | | | | |
| 0.7 mg/140 μl | 0.14 ml | Daily | +++/+++ | Unusable due to # |
| | | (7x/week)(b) | | of injections and |
| | | | | needle damage |
| 1.4 mg/280 μl | 0.28 ml | Every other | +++/+++ | Non-effective, |
| | | day(b) | | many injections |
| 2.5 mg/500 μl | 0.5 ml | 2x/week(c) | +++/++ | Limited tumor |
| | | | | growth |
| 5 mg/1 ml | 1.0 ml | 1x/week | +/−−− | No visible tumor, |
| | | | | well tolerated by |
| | | | | animals |

(a)All animals received the same total dose of VCN of ~5 mg per week (3 animals per group)
(b)Repeated doses required large bore needles, which cause structural leakage in the abdominal wall
(c)Twice weekly limits tumor growth to some extent, but tumor is definitely observable by imaging and visible upon sacrifice

Table 2 shows the dose-response results in SKOV-3^{Luc} model of ovarian cancer. In this experiment, SKOV-3 cells were stably infected with the luciferase gene for the purpose of monitoring tumor growth during the study by optical imaging.

TABLE 2

| VCN Treatment in Oxiplex | Tumor Growth Result^{b} |
|---|---|
| 0 mg/week | ++++ |
| 1 mg/week | ++ |
| 2.5 mg/week | + |
| 5 mg/week | −−− |
| | (none observable) |

^{b}Growth based on a ++++, +++, ++, + and −−−− scale; this corresponds to extensive dissemination and carcinomatosis, less dissemination, some dissemination, minor dissemination and carcinomatosis, no observable tumor dissemination or carcinomatosis. Same tumor growth assessment as in Table 1.

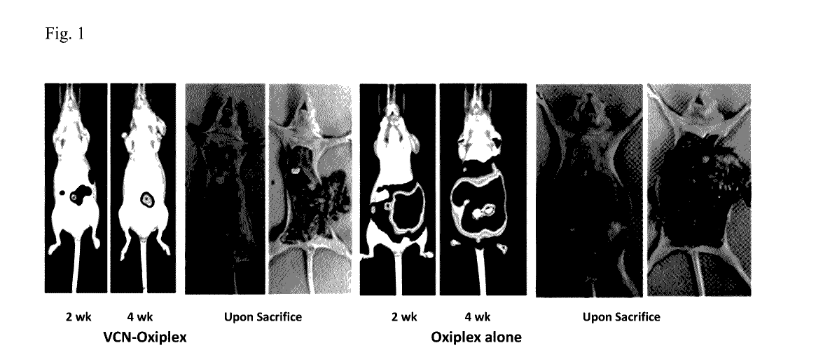

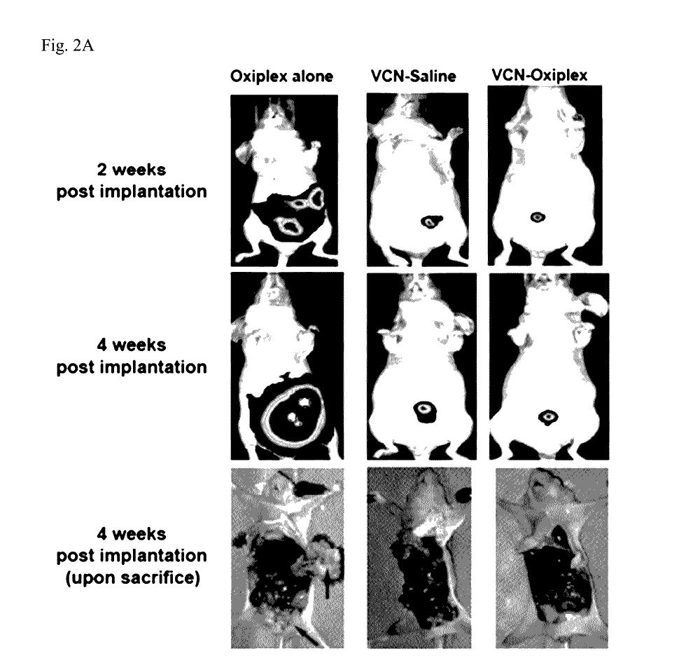

To demonstrate the consistency of the bioluminescent signal put out by the luciferase expressing tumors, recurrent studies in the SKOV3^{GFP/LUC} model in which an inoculum of 2×10⁶ cells was injected IP and the tumors were allowed to grow for 2 weeks prior to the commencement of treatment were carried out. Therapy employed 5 mg VCN delivered in 1 ml Oxiplex once weekly versus 1 ml control Oxiplex alone once weekly for a 4-week course. As shown in FIG. 1, a comparison of representative 2 week- and 4 week-treated animals demonstrates dramatic differences between the groups with the treated animals essentially devoid of visible macroscopic tumor foci by gross examination. Bioluminescent imaging quantitation showed greater than 95% inhibition of tumor spread in the treated groups after 4 weeks of treatment.

### Example 4

### VCN-Oxiplex Vs. VCN-Saline Studies in a Spheroid Model

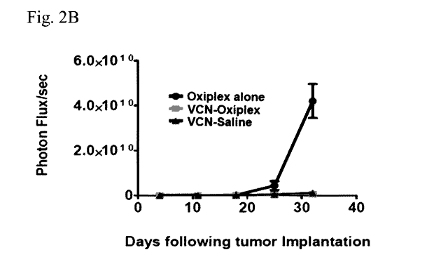

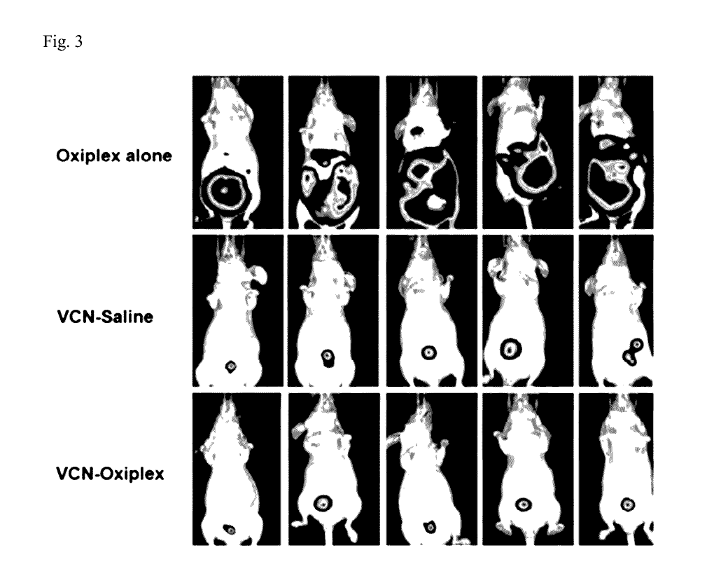

The efficacy of VCN delivered in Oxiplex versus VCN in saline were compared in the SKOV3^{GFP/LUC} spheroid model. The animals (10 animals per group) were inoculated with 0.1 ml of SKOV3^{GFP/LUC} spheroids (2×10⁶ cell equivalent) IP and the spheroids were allowed to implant for 4 days before the treatments were initiated. Once the treatment was started, the animals received IP either a total of 5 mg of VCN delivered in saline every week or 5 mg of VCN in 1 ml of Oxiplex once weekly or 1 ml of Oxiplex alone, also administered once a week. As previously observed, the animals in the Oxiplex alone (control) group grew a large number of carcinomatosis foci disseminated throughout the peritoneal cavity as shown by bioluminescent optical imaging and confirmed by gross examination upon sacrificing. By comparison the animals that received VCN-saline and VCN-Oxiplex treatments showed significantly less or, in some of the animals, no visible macroscopic tumor foci by gross examination (when present, they appeared as much smaller foci of a few mm in diameter), which is consistent with the data that was generated by bioluminescence quantitation (FIGS. 2A & B). The quantitation of the bioluminescent data from this study shows that VCN-Oxiplex inhibits tumor growth by ˜95-98% and VCN-saline by ˜90-95%. These experiments were repeated three times with larger numbers of animals and found reproducible. Reproducibility within the treatment groups is as high as in animal images disclosed in FIG. 3.

These results suggest that both Oxiplex and saline delivery modalities are similarly effective. The Oxiplex formulation is designed with the additional benefit of preventing post-surgical adhesions, which is, as discussed above, a very frequent complication in OC patients. The performance of VCN-Oxiplex with increasing time intervals between injections was evaluated. Data indicates that ascites develops predominantly in the control groups as well as in those groups treated with much lower doses of VCN (i.e., <<5 mg/week).

### Example 5

### VCN does not Alter Coagulation Parameters or Bleeding Times in Mice

Measurement of both Prothrombin Time (PT) and Partial Thromboplastin Time (PTT) reveals that VCN had no effect on either PT or PTT after 3 weeks exposure to VCN in mice. In this study, non-tumor bearing mice were injected IP with 5 mg of VCN in 1 ml Oxiplex once weekly or IV with a total amount of 5 mg of VCN in saline weekly. The determined values fall within the normal values for mice reported in the literature. The average PT was found to be 12 seconds and the PTT 26 seconds (analyses were performed by Charles River Laboratories, Boston, Mass.). Additionally, bleeding times were assessed following IP delivery of VCN-saline or VCN-Oxiplex, or IV delivery of VCN. Mice were injected with weekly therapeutic doses of VCN for two weeks (5 mg of VCN IP in saline or 5 mg of VCN in 1 ml Oxiplex IP weekly or a total amount of 5 mg VCN in saline weekly divided in multiple doses) and compared to untreated control animals. Bleeding time was evaluated after two weeks of continuous treatment by standard methods involving excision of a 0.5 cm portion of the tip of the tail and monitoring bleeding into a beaker containing saline. No significant changes in the measured bleeding times were observed between the animal groups following 2 weeks of VCN therapy: IP VCN-Oxiplex 25 sec, IP VCN-saline 26 sec, IV VCN 26 sec, control 25 sec. These studies indicate that VCN treatment has no effect on platelet function of studied animals.

### Example 6

### Preclinical Toxicity Study in Rats

Toxicity of single IV dose of VCN was evaluated in female Wistar rats (120-130 g). The animal groups (3 animals/group) were: 1 control group (PBS), and 4 experimental groups with VCN (3, 10, 25 and 75 mg/kg for VCN). After a single administration of the agent signs of toxicity or stress were evaluated for 14 days (animals sacrificed on day 14). Toxicity was monitored via physical status, activity and total body weight; gross and microscopic pathologic evaluation was performed and hematological properties were analyzed after animals were sacrificed. There were no adverse effects observed in any treated animals. Animals in all treatment groups thrived and gained weight indistinguishable from control groups. There were no observed changes in behavior immediately after agent administration or during the 14 day study. Gross examination after sacrifice revealed no changes in tissue or organs between control and treated animals. There were no significant differences in hematological parameters between the highest dose (75 mg/kg) and the controls. Microscopic examination of major body organs (lung, liver, kidney, pancreas, eyes, heart and bladder) by a trained pathologist revealed no observable inflammation, no significant cellular alterations and no vascular changes in the microscopic sections.

### Example 7

### Stable Co-Transfection of the OC Cell Lines

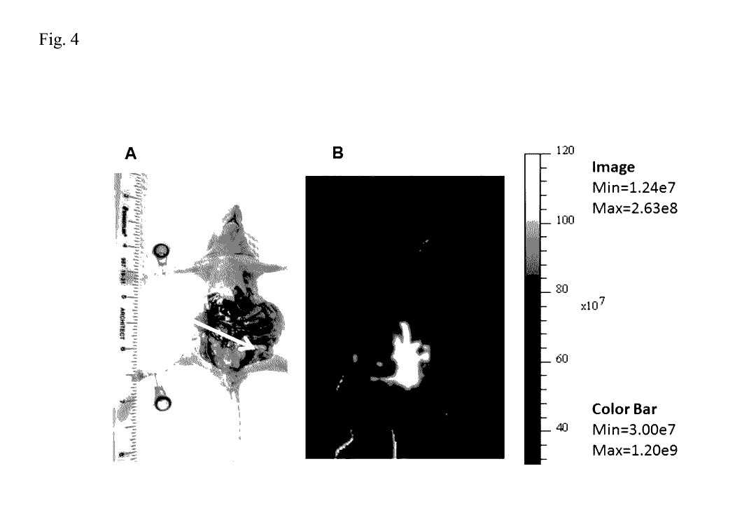

Stable co-transfection of the OC cell lines with a construct containing two genes in series, coding for luciferase (LUC) and GFP, allows real time in vivo monitoring and quantification of the cell burden using the IVIS™ Imaging System (Xenogen, Alameda, Calif., USA) based upon bioluminescence emitted by the product of the gene insert (FIG. 4) as well as post sacrifice evaluation of tumor spread and any metastatic foci using the GFP fluorescence (97). The two genes are arranged in the vector in series separated by an IRES (Internal ribosome entry site) sequence under the control of a constitutive promoter. The transfection using a replication incompetent lentiviral vector allowing for stable integration into the host genome. A bioluminescent signal can be localized in the live mouse and signal intensity in regions of interest can be quantified and correlated with the volume of ovarian cancer cells in the mouse. In the ovarian cancer mouse model studied by Vassileva, LUC transfection of ovarian adenocacinoma cells was verified to be stable over a >2 month period (98). Imaging of the transfected cells by emitted bioluminescence showed high sensitivity and strong correlations between signal, tumor burden and cell viability. In addition, upon termination of the experiment evaluation of tissues by fluorescence microscopy allows for identification of metastatic lesions beyond the primary tumor (24).

### Example 8

### Oxiplex Release Characteristics

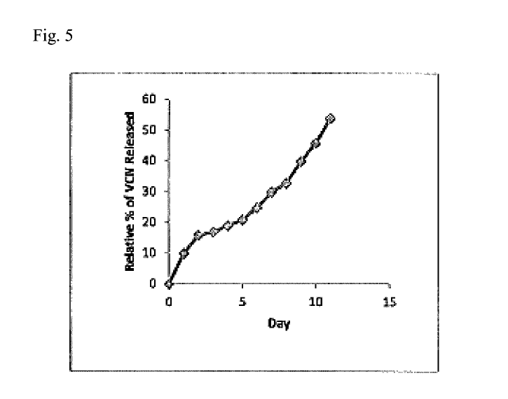

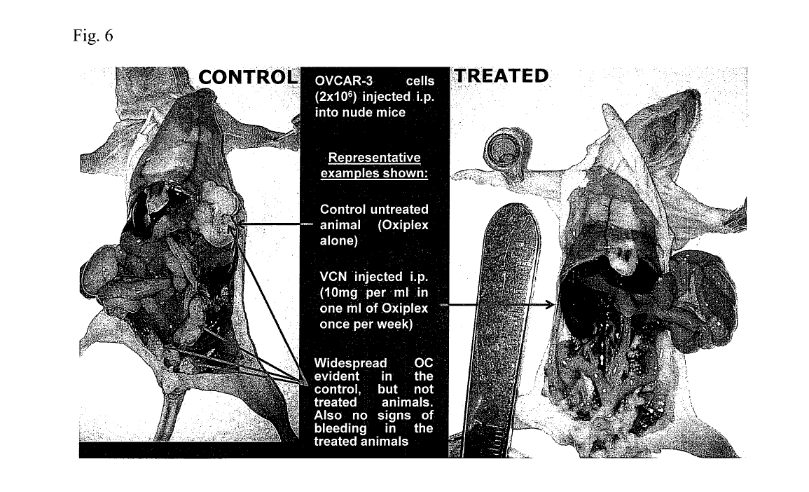

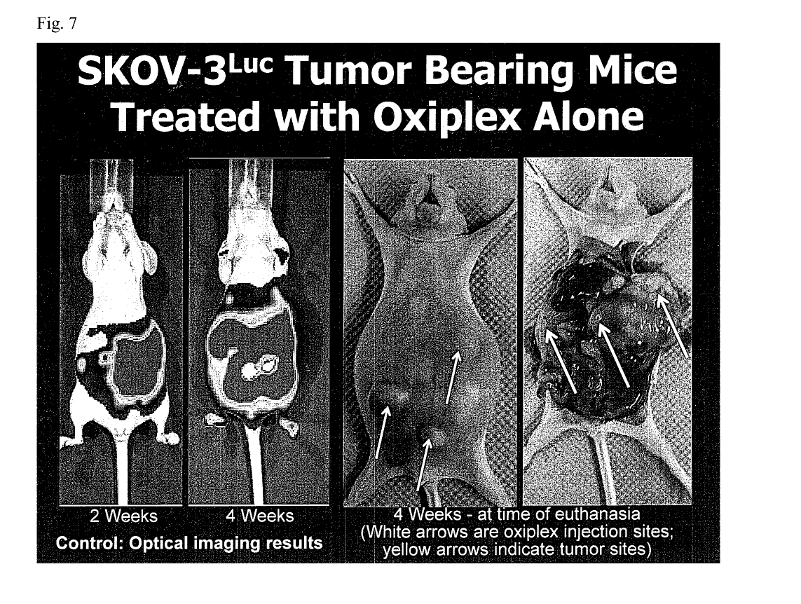

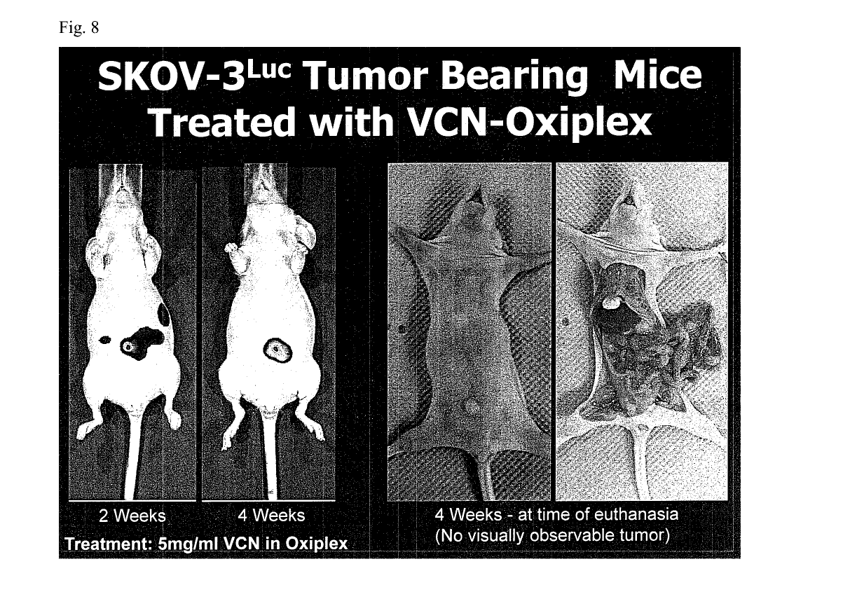

In in vitro studies, Oxiplex releases VCN (as well as other chemotherapeutics) with an initial 2 day burst followed by a linear release over the next 9 days when maintained in a PBS solution at 37° C. (FIG. 5). The level and rate of VCN release can be modulated by alterations in the concentration of VCN as well as the volume of material dispersed into the gel.

In vitro, a VCN impregnated Oxiplex gel will be formed and the release of VCN based on the VCN solution as a percentage of the final gel volume as well as a function of the total amount of VCN loaded in the gel in a fixed volume of 2.5% of the final Oxiplex-VCN volume will be determined. Both of the parameters (% and amount) are critical to the kinetics of release and the structural stability of the gel. As described previously (FIG. 5), the release of VCN and other chemotherapeutics from Oxiplex under steady state conditions has been evaluated: 2 ml of gel containing 2.5% VCN solution (50 μl of 2 mg/ml VCN) was placed in a test tube and covered with 10 ml of PBS. Daily samples of the supernatant were taken (1000) and placed in a 96 well plate and dried. Twelve samples were taken and the volume normalized for samples taken and a quantitative ELISA was performed and the normalized rate and amount of release was determined. These experiments have been repeated three times with the samples described here. In addition the material from the release experiments was evaluated for retention of bioactivity. The released material was evaluated for retention of platelet aggregation inhibitory activity. VCN released from Oxiplex inhibited platelet aggregation with an IC50 identical to that of the native VCN. The released material functions as native protein when released into the IP space. With this data, the concentrations and percentages of VCN impregnated may be evaluated to determine optimal release under these steady state conditions.

### Example 9

### Screening of OC Cell Lines

A panel of human OC cell lines (e.g., OVCAR3, SKOV3, ES2, HEY, A2780, etc.) have been screened for their ability to form spheroids and found that spheroid formation is a universal behavior in OC. Variants of all these cell lines that are stably labeled with GFP/LUC have been prepared. In addition, fluorescently labeled VCN with 3 different fluorophores (FITC, TRITC, and Cy5) have been prepared and tested for binding to a panel of human OC cell lines by FACS analysis. It was found that VCN universally binds to OC cell lines but not to any of the primary mesothelial cells that were also tested (the LP3 and LP9 cells from Coriell Institute, Camden, N.J., or the cells acquired from Zen-Bio, Research Triangle, N.C.). A panel of OC cell lines for integrin expression have been profiled and it was found that mesenchymal αv and α5 integrins are widely expressed by these cells. Variants of these cell lines that are deficient in different αv and α5 integrin combinations have been prepared as they were knocked down with shRNA species via lentivirus constructs that were obtained from Santa Cruz Biotechnology, Dallas, Tex.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

All references cited herein, including those below and including but not limited to all patents, patent applications, and non-patent literature referenced below or in other portions of the specification, are hereby incorporated by reference herein in their entirety.
1. Kanchi, K. L., et al., Integrated analysis of germline and somatic variants in ovarian cancer. Nature communications, 2014. 5: p. 3156.
2. Cooke, S. L., et al., Genomic analysis of genetic heterogeneity and evolution in high-grade serous ovarian carcinoma. Oncogene, 2010. 29(35): p. 4905-13.
3. Network, T. C. G. A. R., Integrated genomic analyses of ovarian carcinoma. Nature, 2011. 474(7353): p. 609-15.
4. Creighton, C. J., et al., Integrated analyses of microRNAs demonstrate their widespread influence on gene expression in high-grade serous ovarian carcinoma. PloS one, 2012. 7(3): p. e34546.
5. Berns, E. M. and D. D. Bowtell, The changing view of high-grade serous ovarian cancer. Cancer research, 2012. 72(11): p. 2701-4.
6. Birrer, M. J., Ovarian cancer: targeting the untargetable. American Society of Clinical Oncology educational book/ASCO. American Society of Clinical Oncology. Meeting, 2014: p. 13-5.
7. Lengyel, E., Ovarian cancer development and metastasis. The American journal of pathology, 2010. 177(3): p. 1053-64.
8. Mitra, A. K., et al., Ligand-independent activation of c-Met by fibronectin and alpha(5)beta(1)-integrin regulates ovarian cancer invasion and metastasis. Oncogene, 2011. 30(13): p. 1566-76.
9. Tomao, F., et al., Investigating molecular profiles of ovarian cancer: an update on cancer stem cells. Journal of Cancer, 2014. 5(5): p. 301-10.
10. Saad, A. F., W. Hu, and A. K. Sood, Microenvironment and pathogenesis of epithelial ovarian cancer. Hormones & cancer, 2010. 1(6): p. 277-90.
11. Sodek, K. L., M. J. Ringuette, and T. J. Brown, Compact spheroid formation by ovarian cancer cells is associated with contractile behavior and an invasive phenotype. Int J Cancer, 2009. 124(9): p. 2060-70.
12. Davidson, B., Biological characteristics of cancers involving the serosal cavities. Crit Rev Oncog, 2007. 13(3): p. 189-227.
13. Jaaback, K., N. Johnson, and T. A. Lawrie, Intraperitoneal chemotherapy for the initial management of primary epithelial ovarian cancer. Cochrane Database Syst Rev, 2011 (11): p. CD005340.
14. Zeimet, A. G., et al., Pros and cons of intraperitoneal chemotherapy in the treatment of epithelial ovarian cancer. Anticancer Res, 2009. 29(7): p. 2803-8.
15. Kwa, M., et al., Ovarian Cancer in BRCA Mutation Carriers: Improved Outcome After Intraperitoneal (IP) Cisplatin. Ann Surg Oncol, 2013.
16. Markman, M., et al., Phase II trial of intraperitoneal paclitaxel in carcinoma of the ovary, tube, and peritoneum: a Gynecologic Oncology Group Study. J Clin Oncol, 1998. 16(8): p. 2620-4.
17. Markman, M., et al., Phase I trial of intraperitoneal taxol: a Gynecoloic Oncology Group study. J Clin Oncol, 1992. 10(9): p. 1485-91.
18. Francis, P., et al., Phase I feasibility and pharmacologic study of weekly intraperitoneal paclitaxel: a Gynecologic Oncology Group pilot Study. J Clin Oncol, 1995. 13(12): p. 2961-7.
19. Markman, M., An update on the use of intraperitoneal chemotherapy in the management of ovarian cancer. Cancer J, 2009. 15(2): p. 105-9.
20. Kwa, M. and F. Muggia, Ovarian cancer: a brief historical overview of intraperitoneal trials. Ann Surg Oncol, 2014. 21(5): p. 1429-34.
21. Armstrong, D. K., et al., Intraperitoneal cisplatin and paclitaxel in ovarian cancer. N Engl J Med, 2006. 354(1): p. 34-43.
22. Vergote, I., et al., Intraperitoneal chemotherapy in patients with advanced ovarian cancer: the con view. Oncologist, 2008. 13(4): p. 410-4.
23. Mackay, H. J., et al., Phase ii/iii study of intraperitoneal chemotherapy after neoadjuvant chemotherapy for ovarian cancer: ncic ctg ov.21. Curr Oncol, 2011. 18(2): p. 84-90.
24. Walker, J. L., et al., Intraperitoneal catheter outcomes in a phase III trial of intravenous versus intraperitoneal chemotherapy in optimal stage III ovarian and primary peritoneal cancer: a Gynecologic Oncology Group Study. Gynecol Oncol, 2006. 100(1): p. 27-32.
25. Markman, M. and J. L. Walker, Intraperitoneal chemotherapy of ovarian cancer: a review, with a focus on practical aspects of treatment. J Clin Oncol, 2006. 24(6): p. 988-94.
26. Rodgers, K. E., et al., Reduction of epidural fibrosis in lumbar surgery with Oxiplex adhesion barriers of carboxymethylcellulose and polyethylene oxide. Spine J, 2003. 3(4): p. 277-83; discussion 284.
27. diZerega, G. S., et al., A modern biomaterial for adhesion prevention. J Biomed Mater Res B Appl Biomater, 2007. 81(1): p. 239-50.
28. Liu, Z. C., et al., Clinical assessment of a CMC/PEO gel to inhibit postoperative epidural adhesion formation after lumbar discectomy: a randomized, control study. Arch Orthop Trauma Surg, 2013. 133(3): p. 295-301.
29. Fransen, P., Safety of carboxymethylcellulose/polyethylene oxide for the prevention of adhesions in lumbar disc herniation-consecutive case series review. Ann Surg Innov Res, 2008. 2: p. 2.
30. Davidson, B., et al., AlphaV-and beta1-integrin subunits are commonly expressed in malignant effusions from ovarian carcinoma patients. Gynecologic oncology, 2003. 90(2): p. 248-57.
31. Goldberg, I., et al., Alphav integrin expression is a novel marker of poor prognosis in advanced-stage ovarian carcinoma. Clinical cancer research: an official journal of the American Association for Cancer Research, 2001. 7(12): p. 4073-9.
32. Casey, R. C., et al., Beta 1-integrins regulate the formation and adhesion of ovarian carcinoma multicellular spheroids. The American journal of pathology, 2001. 159(6): p. 2071-80.
33. Minea, R. O., et al., Vicrostatin—an anti-invasive multi-integrin targeting chimeric disintegrin with tumor anti-angiogenic and pro-apoptotic activities. PLoS One, 2010. 5(6): p. e10929.
34. Schmitmeier, S., et al., Potent mimicry of fibronectin-induced intracellular signaling in glioma cells by the homodimeric snake venom disintegrin contortrostatin. Neurosurgery, 2005. 57(1): p. 141-53; discussion 141-53.
35. Carduner, L., et al., Ovarian cancer ascites-derived vitronectin and fibronectin: combined purification, molecular features and effects on cell response. Biochimica et biophysica acta, 2013. 1830(10): p. 4885-97.
36. Trimble, E. L., et al., Intraperitoneal chemotherapy for women with epithelial ovarian cancer. Oncologist, 2008. 13(4): p. 403-9.
37. Sodek, K. L., et al., Cell-cell and cell-matrix dynamics in intraperitoneal cancer metastasis. Cancer metastasis reviews, 2012. 31(1-2): p. 397-414.
38. Steinkamp, M. P., et al., Ovarian tumor attachment, invasion, and vascularization reflect unique microenvironments in the peritoneum: insights from xenograft and mathematical models. Frontiers in oncology, 2013. 3: p. 97.
39. Shield, K., et al., Multicellular spheroids in ovarian cancer metastases: Biology and pathology. Gynecologic oncology, 2009. 113(1): p. 143-8.
40. Iwanicki, M. P., et al., Ovarian cancer spheroids use myosin-generated force to clear the mesothelium. Cancer discovery, 2011. 1(2): p. 144-57.
41. Schwarzbauer, J. E. and D. W. DeSimone, Fibronectins, their fibrillogenesis, and in vivo functions. Cold Spring Harbor perspectives in biology, 2011. 3(7).
42. Davidson, B., et al., αV-and β1-integrin subunits are commonly expressed in malignant effusions from ovarian carcinoma patients. Gynecologic oncology, 2003. 90(2): p. 248-257.
43. Casey, R. C., et al., β1-Integrins Regulate the Formation and Adhesion of Ovarian Carcinoma Multicellular Spheroids. The American Journal of Pathology, 2001. 159(6): p. 2071-2080.
44. Kellouche, S., et al., Initial formation of IGROV1 ovarian cancer multicellular aggregates involves vitronectin. Tumour Biol, 2010. 31(2): p. 129-39.
45. Sawada, K., et al., Loss of E-cadherin promotes ovarian cancer metastasis via alpha 5-integrin, which is a therapeutic target. Cancer research, 2008. 68(7): p. 2329-39.
46. Sawada, K., et al., Integrin inhibitors as a therapeutic agent for ovarian cancer. Journal of oncology, 2012. Article ID 915140.
47. Almokadem, S. and C. P. Belani, Volociximab in cancer. Expert opinion on biological therapy, 2012. 12(2): p. 251-7.
48. Chen, Y. Q., et al., Ectopic expression of platelet integrin alphallb beta3 in tumor cells from various species and histological origin. Int J Cancer, 1997. 72(4): p. 642-8.
49. Askari, J. A., et al., Linking integrin conformation to function. J Cell Sci, 2009. 122(Pt 2): p. 165-70.
50. Hynes, R. O., Integrins: versatility, modulation, and signaling in cell adhesion. Cell, 1992. 69(1): p. 11-25.
51. Pignatelli, M., et al., Integrins and their Accessory Adhesion Molecules in Mammary Carcinomas: Loss of Polarization in Poorly Differentiated Tumors. Human Pathology, 1992. 23: p. 1159-1166.
52. Ruoslahti, E., Integrins. J Clin Invest, 1991. 87(1): p. 1-5.
53. Hapke, S., et al., Ovarian cancer cell proliferation and motility is induced by engagement of integrin alpha(v)beta3/Vitronectin interaction. Biol Chem, 2003. 384(7): p. 1073-83.
54. Gillan, L., et al., Periostin secreted by epithelial ovarian carcinoma is a ligand for alpha(V)beta(3) and alpha(V)beta(5) integrins and promotes cell motility. Cancer Res, 2002. 62(18): p. 5358-64.
55. Ahmed, N., et al., Overexpression of alpha(v)beta6 integrin in serous epithelial ovarian cancer regulates extracellular matrix degradation via the plasminogen activation cascade. Carcinogenesis, 2002. 23(2): p. 237-44.
56. Cannistra, S. A., et al., Expression and function of beta 1 and alpha v beta 3 integrins in ovarian cancer. Gynecol Oncol, 1995. 58(2): p. 216-25.
57. Swenson, S., S. Ramu, and F. S. Markland, Anti-angiogenesis and RGD-containing snake venom disintegrins. Curr Pharm Des, 2007. 13(28): p. 2860-71.
58. McLane, M. A., T. Joerger, and A. Mahmoud, Disintegrins in health and disease. Front Biosci, 2008. 13: p. 6617-37.
59. Gould, R. J., et al., Disintegrins: a family of integrin inhibitory proteins from viper venoms. Proc Soc Exptl Biol Med, 1990. 195(2): p. 168-71.
60. Saudek, V., R. A. Atkinson, and J. T. Pelton, Three-Dimensional Structure of Echistatin, the Smallest Active RGD Protein. Biochemistry, 1991. 30: p. 7369-7372.
61. Moiseeva, N., et al., Structure of acostatin, a dimeric disintegrin from Southern copperhead (Agkistrodon contortrix contortrix), at 1.7A resolution. Acta Crystallogr D Biol Crystallogr, 2008. 64(Pt 4): p. 466-70.
62. Shebuski, R. J., et al., Characterization and platelet inhibitory activity of bitistatin, a potent arginine-glycine-aspartic acid-containing peptide from the venom of the viper Bitis arietans. J Biol Chem, 1989. 264(36): p. 21550-6.
63. Yasuda, T., et al., Kistrin, a polypeptide platelet GPIIb/IIIa receptor antagonist, enhances and sustains coronary arterial thrombolysis with recombinant tissue-type plasminogen activator in a canine preparation. Circulation, 1991. 83(3): p. 1038-47.
64. Cousins, G. R., et al., Contortrostatin Prevents Reocclusion after Thrombolytic Therapy in a Canine Model of Carotid Artery Thrombosis. FASEB J., 1995. 9: p. A938.
65. Sheldrake, H. M. and L. H. Patterson, Strategies To Inhibit Tumor Associated Integrin Receptors: Rationale for Dual and Multi-Antagonists. J Med Chem, 2014.
66. Sanz, L., et al., cDNA cloning and functional expression of jerdostatin, a novel RTS-disintegrin from Trimeresurus jerdonii and a specific antagonist of the alpha1beta1 integrin. J Biol Chem, 2005. 280(49): p. 40714-22.
67. Brown, M. C., et al., Structural requirements of KTS-disintegrins for inhibition of alpha(1)beta(1) integrin. Biochem J, 2009. 417(1): p. 95-101.
68. Marcinkiewicz, C., et al., Significance of RGD loop and C-terminal domain of echistatin for recognition of alphallb beta3 and alpha(v) beta3 integrins and expression of ligand-induced binding site. Blood, 1997. 90(4): p. 1565-75.
69. Fujii, Y., et al., Crystal structure of trimestatin, a disintegrin containing a cell adhesion recognition motif RGD. J Mol Biol, 2003. 332(5): p. 1115-22.
70. Calvete, J. J., et al., Snake venom disintegrins: evolution of structure and function. Toxicon, 2005. 45(8): p. 1063-74.
71. Yahalom, D., et al., Identification of the principal binding site for RGD-containing ligands in the alpha(V)beta(3) integrin: a photoaffinity cross-linking study. Biochemistry, 2002. 41(26): p. 8321-31.
72. Kini, R. M., Toxins in thrombosis and haemostasis: potential beyond imagination. J Thromb Haemost, 2011. 9 Suppl 1: p. 195-208.
73. Hirschhaeuser, F., et al., Multicellular tumor spheroids: an underestimated tool is catching up again. Journal of biotechnology, 2010. 148(1): p. 3-15.
74. Vinci, M., et al., Advances in establishment and analysis of three-dimensional tumor spheroid-based functional assays for target validation and drug evaluation. BMC biology, 2012. 10: p. 29.
75. Burleson, K. M., et al., Ovarian carcinoma ascites spheroids adhere to extracellular matrix components and mesothelial cell monolayers. Gynecologic oncology, 2004. 93(1): p. 170-81.
76. Burleson, K. M., L. K. Hansen, and A. P. N. Skubitz, Ovarian carcinoma spheroids disaggregate on type I collagen and invade live human mesothelial cell monolayers. Clinical & experimental metastasis, 2005. 21(8): p. 685-697.
77. Han, E. S., M. Wakabayashi, and L. Leong, Angiogenesis inhibitors in the treatment of epithelial ovarian cancer. Curr Treat Options Oncol, 2013. 14(1): p. 22-33.
78. Muggia, F., Bevacizumab in ovarian cancer: unanswered questions. Drugs, 2012. 72(7): p. 931-6.
79. Huang, G. S., et al., Potentiation of taxol efficacy and by discodermolide in ovarian carcinoma xenograft-bearing mice. Clin Cancer Res, 2006. 12(1): p. 298-304.
80. Jandial, D. D., et al., Tumor platinum concentration following intraperitoneal administration of cisplatin versus carboplatin in an ovarian cancer model. Gynecol Oncol, 2009. 115(3): p. 362-6.
81. Rein, D. T., et al., Systemic administration of bevacizumab prolongs survival in an in vivo model of platinum pre-treated ovarian cancer. Oncol Lett, 2012. 3(3): p. 530-534.
82. Vassileva, V., C. J. Allen, and M. Piquette-Miller, Effects of sustained and intermittent paclitaxel therapy on tumor repopulation in ovarian cancer. Mol Cancer Ther, 2008. 7(3): p. 630-7.
83. Kumar, C. C., Integrin alpha v beta 3 as a therapeutic target for blocking tumor-induced angiogenesis. Curr Drug Targets, 2003. 4(2): p. 123-31.
84. Cal, W. and X. Chen, Anti-angiogenic cancer therapy based on integrin alphavbeta3 antagonism. Anticancer Agents Med Chem, 2006. 6(5): p. 407-28.
85. Ma, W. W. and A. A. Adjei, Novel agents on the horizon for cancer therapy. CA Cancer J Clin, 2009. 59(2): p. 111-37.
86. Avraamides, C. J., B. Garmy-Susini, and J. A. Varner, Integrins in angiogenesis and lymphangiogenesis. Nat Rev Cancer, 2008. 8(8): p. 604-17.
87. Desgrosellier, J. S. and D. A. Cheresh, Integrins in cancer: biological implications and therapeutic opportunities. Nat Rev Cancer, 2010. 10(1): p. 9-22.
88. Hood, J. D. and D. A. Cheresh, Role of integrins in cell invasion and migration. Nat Rev Cancer, 2002. 2(2): p. 91-100.
89. Schottelius, M., B. Laufer, H. Kessler, and H. J. Wester, Ligands for mapping alphavbeta3-integrin expression in vivo. Acc Chem Res, 2009. 42(7): p. 969-80.
90. Zhou, Y., S. Chakraborty, and S. Liu, Radiolabeled Cyclic RGD Peptides as Radiotracers for Imaging Tumors and Thrombosis by SPECT. Theranostics, 2011. 1: p. 58-82.
91. Trikha, M., Y. A. De Clerck, and F. S. Markland, Contortrostatin, a snake venom disintegrin, inhibits beta 1 integrin-mediated human metastatic melanoma cell adhesion and blocks experimental metastasis. Cancer Res, 1994. 54(18): p. 4993-8.
92. Jameson, D. M. and G. Mocz, Fluorescence polarization/anisotropy approaches to study protein-ligand interactions: effects of errors and uncertainties. Methods Mol Biol, 2005. 305: p. 301-22.
93. Shaw, T. J., M. K. Senterman, K. Dawson, C. A. Crane, and B. C. Vanderhyden, Characterization of intraperitoneal, orthotopic, and metastatic xenograft models of human ovarian cancer. Mol Ther, 2004. 10(6): p. 1032-42.
94. Hamilton, T. C., R. C. Young, and R. F. Ozols, Experimental model systems of ovarian cancer: applications to the design and evaluation of new treatment approaches. Semin Oncol, 1984. 11(3): p. 285-98.
95. Bao, R., M. Selvakumaran, and T. C. Hamilton, Use of a surrogate marker (human secreted alkaline phosphatase) to monitor in vivo tumor growth and anticancer drug efficacy in ovarian cancer xenografts. Gynecol Oncol, 2000. 78(3 Pt 1): p. 373-9.
96. Swenson, S., F. Costa, W. Ernst, G. Fujii, and F. S. Markland, Contortrostatin, a snake venom disintegrin with anti-angiogenic and anti-tumor activity. Pathophysiol Haemost Thromb, 2005. 34(4-5): p. 169-76.
97. Duiker, E. W., E. G. de Vries, D. Mahalingam, G. J. Meersma, W. Boersma-van Ek, H. Hollema, M. N. Lub-de Hooge, G. M. van Dam, R. H. Cool, W. J. Quax, A. Samali, A. G. van der Zee, and S. de Jong, Enhanced antitumor efficacy of a DR5-specific TRAIL variant over recombinant human TRAIL in a bioluminescent ovarian cancer xenograft model. Clin Cancer Res, 2009. 15(6): p. 2048-57.
98. Vassileva, V., E. H. Moriyama, R. De Souza, J. Grant, C. J. Allen, B. C. Wilson, and M. Piquette-Miller, Efficacy assessment of sustained intraperitoneal paclitaxel therapy in a murine model of ovarian cancer using bioluminescent imaging. Br J Cancer, 2008. 99(12): p. 2037-43.
99. Davies, G., J. Salter, M. Hills, L. A. Martin, N. Sacks, and M. Dowsett, Correlation between cyclooxygenase-2 expression and angiogenesis in human breast cancer. Clin Cancer Res, 2003. 9(7): p. 2651-6.
100. Mullen, P. and S. P. Langdon, The use of matrigel in the establishment of ovarian carcinoma cell lines as xenografts. Methods Mol Med, 2001. 39: p. 199-203.
101. U.S. Pat. No. 7,754,850.
102. U.S. Pat. No. 8,110,542.
103. U.S. Pat. No. 8,338,365.
104. U.S. Pat. No. 5,906,997.

What is claimed is:

1. A method of treating ovarian cancer (OC), comprising:
administering to a subject in need thereof an effective amount of a pharmaceutical composition comprising a protein comprising SEQ ID NO: 1 and/or a protein comprising a polypeptide having an amino acid sequence substantially the same as SEQ ID NO: 1.

2. The method of claim 1, wherein the pharmaceutical composition comprises a protein comprising SEQ ID NO: 1.

3. The method of claim 2, wherein the pharmaceutical composition further comprises saline or phosphate buffered saline.

4. The method of claim 2, wherein the pharmaceutical composition comprises the protein loaded in a viscoelastic gel.

5. The method of claim 4, wherein the viscoelastic gel comprises polyethylene oxide (PEO) and carboxymethyl cellulose (CMC).

6. The method of claim 2, wherein the administering is performed intraperitoneally.

7. The method of claim 2, further comprising concurrently or sequentially administering to the patient one or more additional treatments for ovarian cancer, wherein the one or more additional treatments does not include a disintegrin.

8. The method of claim 7, wherein the additional treatment comprises performing a debulking surgery comprising a surgical incision permitting access to the peritoneal space of the subject, and administration of the pharmaceutical composition is performed after the debulking but before the close of the surgical incision.

9. The method of claim 7, wherein the additional treatment comprises one or more selected from the group consisting of chemotherapy, immune and radiation therapy.

10. A method of treating ovarian cancer comprising intraperitoneally administering to a subject in need an effective amount of a pharmaceutical composition comprising a disintegrin loaded in a viscoelastic gel, wherein the disintegrin comprises one or more selected from the group consisting of a protein comprising SEQ ID NO: 1 and a protein comprising a polypeptide substantially the same as SEQ NO: 1.

11. The method of claim 10, wherein the disintegrin is a protein comprising SEQ ID NO: 1.

12. The method of claim 11, wherein the viscoelastic gel comprises polyethylene oxide (PEO) and carboxymethyl cellulose (CMC).

13. The method of claim 11, further comprising concurrently or sequentially administering to the patient one or more additional treatments for ovarian cancer, wherein the one or more additional treatments does not include a disintegrin.

14. The method of claim 13, wherein the additional treatment comprises performing a debulking surgery comprising a surgical incision permitting access to the peritoneal space of the patient, and administration of the pharmaceutical composition is performed after the debulking but before the close of the surgical incision.

15. The method of claim 14, wherein the additional treatment comprises one or more selected from the group consisting of chemotherapy, immune and radiation therapy.

16. A pharmaceutical composition comprising a disintegrin loaded in a viscoelastic gel comprising polyethylene oxide (PEO) and carboxymethyl cellulose (CMC), wherein the disintegrin comprises one or more of selected from the group consisting of a protein comprising SEQ ID NO: 1 and a protein comprising a polypeptide having an amino acid sequence substantially the same as SEQ ID NO: 1.

17. The pharmaceutical composition of claim 16, wherein the disintegrin is a protein comprising SEQ ID NO: 1.

18. The method of claim 17, wherein the disintegrin loaded PEO/CMC gel is prepared by combining a volume of the PEO/CMC gel diluted less than 20% by a volume of the disintegrin.

19. The method of claim 17, wherein the disintegrin loaded PEO/CMC gel is prepared by combining a volume of the PEO/CMC gel diluted less than 5% by a volume of the disintegrin.
